(19) United States
(12) Patent Application Publication
(10) Pub. No.: US 2026/0092855 A1
(43) Pub. Date: Apr. 2, 2026

(54) OPTICAL DETERMINATION OF WHITE BLOOD CELL CONCENTRATION IN FLUIDS

(71) Applicant: Gambro Lundia AB, Lund (SE)

(72) Inventors: Mattias Holmer, Lund (SE); Matilda Eva Träff, Sodra Sandby (SE); Hiba Zohra Shahid, Vernon Hills, IL (US); Per-Olof Borgqvist, Lund (SE); Anders O. Wallenborg, Bjarred (SE); James McCanna, Deerfield, IL (US); Shawn Collin Oppegard, Fox River Grove, IL (US); Jorge Augusto Del Castillo, Des Plaines, IL (US)

(21) Appl. No.: 18/904,869

(22) Filed: Oct. 2, 2024

(51) Int. Cl. G01N 15/075; A61M 1/28

(52) U.S. Cl. CPC G01N 15/075; A61M 1/28; A61M 2205/3313; A61M 2230/20

(57) ABSTRACT

The concentration of white blood cells in a fluid is determined by an optical detection apparatus. The apparatus comprises a light emitting arrangement for generating a light beam with a wavelength in the range of 350-575 nm, and a light detection arrangement for detecting scattered light from a thus-illuminated region in the fluid. A computing apparatus determines a plurality of properties of the scattered light, and operates a calculation function on the plurality of properties to estimate the concentration of white blood cells in the fluid. The computing apparatus may additionally determine and use a property of the light of the light beam that is transmitted by the fluid and/or a property of light that is scattered by the fluid from a second light beam with a wavelength in the range of 600-1000 nm.

### TECHNICAL FIELD

The present disclosure relates generally to optical techniques for measuring density of particles in fluids, and in particular the concentration of white blood cells in effluent from peritoneal dialysis.

### BACKGROUND ART

Inflammation in the peritoneum is common among patients undergoing peritoneal dialysis (PD). Early detection of infection or inflammation (“peritonitis”) is essential to avoid suffering and therapy drop-out. Basically, there are two modalities for carrying out PD: automated peritoneal dialysis (APD) and a manual non-automated procedure denoted continuous ambulatory peritoneal dialysis (CAPD). In CAPD, infection may be detected by visual inspection of effluent bags in which spent dialysis fluid (“effluent”) is collected. A cloudy effluent bag is a sign of peritonitis. The cloudiness is caused by increased presence of white blood cells (WBCs) caused by the infection. In APD, the effluent is often passed through an effluent line directly to the drain, and no visual inspection is possible. The infection is therefore detected late, when other signs such as stomach pain appear, and the peritoneum may be damaged.

It may also be relevant to detect presence of red blood cells (RBCs) in the effluent, since RBCs is a sign of bleeding within the peritoneal cavity (hemoperitoneum). The RBCs may originate from the peritoneal membrane, from the intraperitoneal organs (organs that are fully encapsulated by the visceral peritoneal membrane), or from partially or completely extraperitoneal structures. For women, the RBCs may be caused by normal ovulation or menstruation. If this origin can be ruled out, the RBCs may be a sign of issues with the PD catheter, or a more acute medical condition such as splenic laceration, liver rupture, liver or renal cyst rupture, erosion of mesenteric vessel, bleeding from a malignant tumor, etc.

The prior art comprises WO2022/008213, which discloses a technique of illuminating a fluid with a light beam and detecting scattered and/or transmitted light, where the particle density in the fluid is given by the temporal variability of the scattered and/or transmitted light. The technique is useful for determining the concentration of WBCs in PD effluent, presuming that the particles are WBCs.

WO2019/118929 proposes a technique of illuminating PD effluent with a light beam and detecting scattered and/or transmitted light, where the particle density in the PD effluent is given by the magnitude of the scattered and/or transmitted light. It is also proposed to use two light beams for the illumination to discriminate between RBCs and WBCs; a first light beam in the infrared (IR), and a second light beam in the range of 260-550 nm. A magnitude value of the scattered and/or transmitted light is determined for the first beam and second beam, respectively. The proposed evaluation technique results in a concentration value of WBCs or RBCs in the PF effluent and uses a first correlation plot that relates magnitude values for the first beam to concentration of RBCs and WBCs, respectively, and a second correlation plot that relates the ratio of magnitude values for the first and second beams to the concentration of RBCs and WBCs, respectively. As far can be understood, the proposed evaluation technique presumes that the effluent contains either WBCs or RBCs.

### SUMMARY

It is an objective to at least partly overcome one or more limitations of the prior art.

One objective is to provide an optical technique for estimating the concentration of white blood cells (WBCs) in a fluid.

Another objective is to provide such a technique that is capable of estimating the concentration of WBCs in the presence of red blood cells (RBCs).

A further objective is to provide such a technique that is capable of estimating the concentration of RBCs in the presence of WBCs.

One or more of these objectives, as well as further objectives that may appear from the description below, are at least partly achieved by an optical detection apparatus according to the independent claim, embodiments thereof being defined by the dependent claims.

The present disclosure emanates from a significant experimental effort by the Applicant to understand the dependence of scattered and transmitted light from a fluid, when illuminated by light beams at different wavelengths, on the concentration of WBCs and RBCs in the fluid. Surprisingly, the Applicant has found that the concentration of WBCs in a fluid can be estimated, even if the fluid contains RBCs, by use of a first time-dependent signal from a detector arranged to receive scattered light from the fluid when illuminated by a light beam with a wavelength in the range of 350-575 nm. According to the Applicant's findings, it is possible to correctly estimate the concentration of WBCs based on two or more properties that represent the first time-dependent signal. In this context, a correct estimation of WBC concentration lies within +20% of the ground truth or +100 cells/μL, whichever is the largest. The Applicant has also identified further time-dependent signals that may be used to improve the estimation.

Further, the Applicant has found that the combined concentration of WBCs and RBCs in a fluid can be estimated, by use of the first time-dependent signal in combination with a second time-dependent signal from a detector arranged to receive scattered light from the fluid when illuminated by a light beam with a wavelength in the range of 600-1000 nm. When the combined concentration has been estimated, the concentration of RBCs in the fluid can be estimated by accounting for the estimated concentration of WBCs.

Still other objectives as well as embodiments, features, advantages and technical effects may appear from the following detailed description, from the attached claims as well as from the drawings.

### BRIEF DESCRIPTION OF THE DRAWINGS

FIG. 1A is a side view of an example system for peritoneal dialysis comprising an optical detection apparatus, and FIG. 1B is a side view, partly in section, of an example optical detection apparatus.

FIGS. 2A-2C are side views of example arrangements for detection of transmitted and scattered light in relation to a fluid.

FIGS. 3A-3B are graphs of an example signal representing scattered light after normalization.

FIGS. 4A-4B are graphs of an example signal representing transmitted light after normalization.

FIGS. 5A-5B are histograms corresponding to the signals in FIG. 3A and FIG. 4A.

FIG. 6 is a graph of optical absorption spectra for hemoglobin.

FIG. 7 is a block diagram of an example device for determining densities of white blood cells (WBCs) and red blood cells (RBCs) in a fluid.

FIG. 8A is a flow chart of an example method of determining the concentration of WBCs in a fluid, and FIG. 8B is a flow chart of an example method of determining the concentration of RBCs in the fluid.

FIGS. 9A-9B are block diagrams of example devices for determining the concentration of WBCs and the total concentration of cells, respectively, in a fluid.

FIG. 10A shows the average (top) and the standard deviation (bottom) of detected light from a WBC-containing fluid upon illumination by a laser beam at 650 nm as a function of detection angle, and FIG. 10B shows the average (top) and the standard deviation (bottom) of detected light from an RBC-containing fluid upon illumination by a laser beam at 650 nm as a function of detection angle.

FIG. 11A shows the average (top) and the standard deviation (bottom) of detected light from a WBC-containing fluid upon illumination by a laser beam at 405 nm as a function of detection angle, and FIG. 11B shows the average (top) and the standard deviation (bottom) of detected light from an RBC-containing fluid upon illumination by a laser beam at 405 nm as a function of detection angle.

FIG. 12 is a graph of an estimation error as a function of the number of properties used to estimate total cell concentration in accordance with an example.

FIGS. 13-14 are graphs of estimated WBC concentration versus actual WBC concentration for a first estimation example and a second estimation example, respectively.

FIGS. 15-16 are graphs of estimated total cell concentration versus actual total cell concentration for a first estimation example and a second estimation example, respectively.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments. The term “and/or” includes any and all combinations of one or more of the associated listed items.

It will furthermore be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

Like reference signs refer to like elements throughout.

Embodiments will be described with reference to deployment in conjunction with automated peritoneal dialysis (“APD”) therapy. In peritoneal dialysis (PD), dialysis fluid is infused into a patient's peritoneal cavity. This cavity is lined by the peritoneal membrane (“peritoneum”) which is highly vascularized. Substances are removed from the patient's blood by diffusion across the peritoneum into the dialysis fluid. Excess fluid (water) is also removed by osmosis induced by a hypertonic dialysis fluid. Automated peritoneal dialysis (“APD”) is performed by an APD machine, commonly known as a “cycler”. The cycler is operable to automatically perform one or more treatment cycles including fill, dwell and drain phases, for example while the patient sleeps. The cycler is fluidly connected to an implanted catheter, to a source of dialysis fluid and to a fluid drain. The cycler is operated to pump fresh dialysis fluid from the source, through the catheter, into the patient's peritoneal cavity and to allow the dialysis fluid to dwell within the cavity for the transfer of waste, toxins and excess water to take place. The cycler is then operated to pump spent dialysis fluid from the peritoneal cavity, through the catheter, to the drain. Spent dialysis fluid is commonly known as “effluent”.

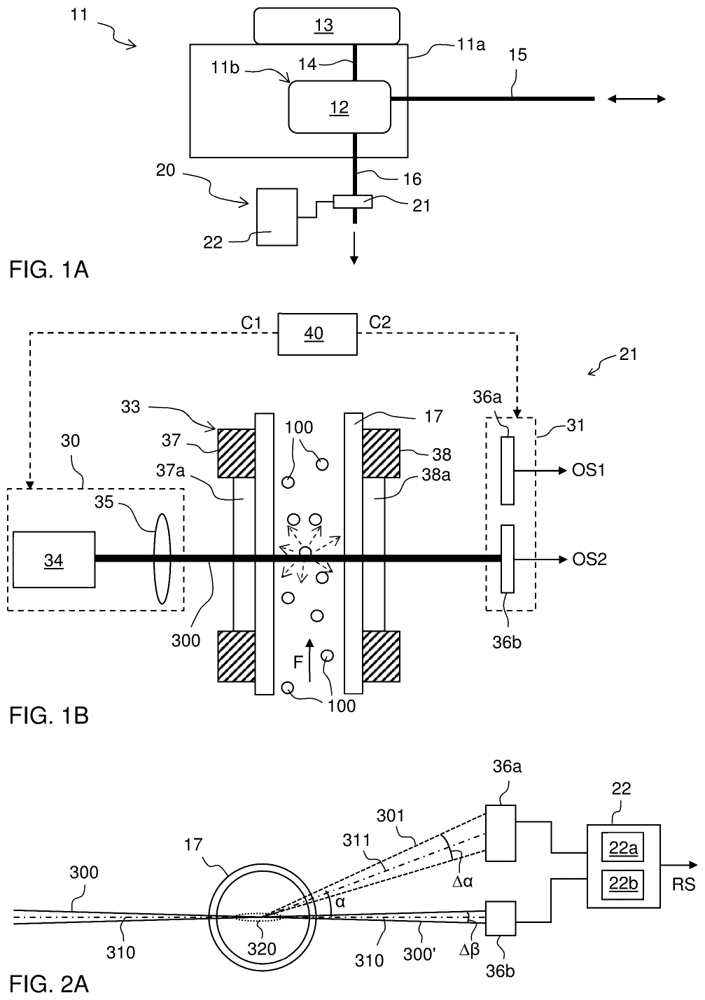

FIG. 1 is a schematic elevated side view of an arrangement 11 for peritoneal dialysis. The arrangement 11 comprises an APD machine 11a (“cycler”). The arrangement further comprises a disposable unit 11b mounted onto the cycler 11a. The cycler 11a comprises a combination of a control system, sensors and actuators to properly move fluid inside a hydraulic circuit of the disposable unit 11b. Although not shown in FIG. 1, the cycler 11a may also comprise a user interface for input/output of data. The disposable unit 11b comprises a cassette 12, as well as a set of tubes (“tubing set”) connected to the cassette 12. In the illustrated example, the tubing set includes a container line 14 terminating in a container 13 configured for holding a treatment fluid (“dialysis fluid”). The container 13 may be in the form of a collapsible bag to be positioned, for example, in a dedicated tray on the cycler 11a. The container 13 may be delivered as a ready-made bag of dialysis fluid, or the container 13 may be filled by dialysis fluid prepared on-line by the cycler 11a or a separate machine (not shown), for example by mixing one or more concentrates with water. The cycler 11a may comprise a heater (not shown) for heating the treatment fluid before it is supplied to the patient. The tubing set further comprises a patient line 15 for connection to a catheter (not shown) implanted in the patient, and a drain line 16 for dispensing spent treatment fluid (“effluent”) to a drain (not shown), for example a container or a sink. During operation, the arrangement 11 performs a fill phase in which a pumping mechanism in the cycler 11a actuates the cassette 12 to pump treatment fluid from the container 13 to the patient through the lines 14, 15, a dwell phase in which the treatment fluid is left within the peritoneal cavity of the patient, and a drain phase in which spent treatment fluid (effluent) is pumped to the drain through the patient and drain lines 15, 16.

Patients on PD are exposed to an elevated risk of attracting infection or inflammation in the peritoneal cavity, caused by bacteria admitted via the indwelling catheter. Often such infection or inflammation is located at the peritoneum and is denoted “peritonitis”. Developed peritonitis may be manifested by the patient experiencing fever, diffuse abdominal pain, and nausea. Peritonitis represents a medical emergency, and early detection and treatment is essential to reduce morbidity and mortality in PD patients. In addition, repeated episodes of peritonitis may contribute to vascular proliferation and interstitial fibrosis, with ensuing loss of ultrafiltration capacity and therapy failure. In PD, peritonitis may be detected by extracting and analyzing the concentration of white blood cells (WBCs) in the effluent. According to established practice, a WBC concentration above 100 cells/μL in the effluent of PD is regarded as a sign of peritonitis and may result in the patient being given antibiotics.

The present disclosure relates to techniques that enable early detection of peritonitis in patients undergoing PD without the need to extract and analyze samples of the effluent. This is achieved by use of an optical detection apparatus (OPA) for mounting on the drain line. The OPA is thereby operable to produce a signal representative of the WBC concentration in the effluent that flows through the drain line.

In FIG. 1, the OPA is represented by reference numeral 20 and comprises a measurement device 21, which is mounted onto the drain line 16, and a computing apparatus 22 which is configured to receive and process one or more output signals of the measurement device 21 for determination of a value representative of the WBC concentration. It is conceivable that the measurement device and computing apparatus 22 are integrated in a single unit. In a variant, the measurement device 21 is configured to wirelessly transmit its output signal(s) to the computing apparatus 22, which thus may be located remotely from the measurement device 21. In a further variant, the measurement device 21 and/or the computing apparatus 22 is included in the cycler 11a. It is also conceivable that the measurement device 21 is mounted onto the patient line 15.

In some embodiments, the measurement device 21 is mounted onto a “tubing portion” which is transparent or at least translucent. The tubing portion may be included in the drain line 16, as shown in FIG. 1, or in the patient line 15. As used herein, a “transparent” material has the property of transmitting light with little scattering so that objects are clearly visible through the material, and a “translucent” material has the property of permitting passage of light while diffusing it so that objects are not clearly visible through the material. Commonly, the tubings in the disposable 11b are made of transparent or translucent material. The tubing portion may thus be an integral part of the drain line 16 or patient line 15. However, should the line 16/15 be made of opaque material, the tubing portion may be a separate tubing of transparent or translucent material that is spliced into the line 16/15. However, it is also conceivable that the tubing portion is a specialized cavity or chamber that is installed in the line 16/15 to facilitate the measurements by measurement device 21. Such a specialized cavity may be or comprise a cuvette with planar optical surfaces of transparent material.

In some embodiments, the OPA 20 is arranged to communicate with the cycler 11a. For example, the OPA 20 may receive, from the cycler 11a, a signal that indicates start of a drain phase and thereby presence of effluent in the drain line 16. Alternatively or additionally, the signal from the cycler 11a may trigger the OPA 20 to measure the WBC concentration. It is also conceivable that the OPA 20 transmits a signal indicative of the measurement result to the cycler 11a, which may operate a feedback unit to inform a user thereof.

In some embodiments, the OPA 20 is configured to illuminate a sample of effluent by light at one or more wavelengths, detect light that is scattered by the sample and/or light that is transmitted through the sample as a result of the illumination, and analyze the detected light for calculation of an output value that represents the concentration of WBCs in the effluent. For practical use, it is desirable for the OPA 20 to properly estimate the concentration of WBCs even if the effluent comprises other types of particles. One type of particle that is seen as problematic in this context is red blood cells (RBCs), which are known to interact with light by both absorption and scattering. As described in the Background section, RBCs may be present in the effluent for natural reasons (menstruation) or as a result of a complication or illness.

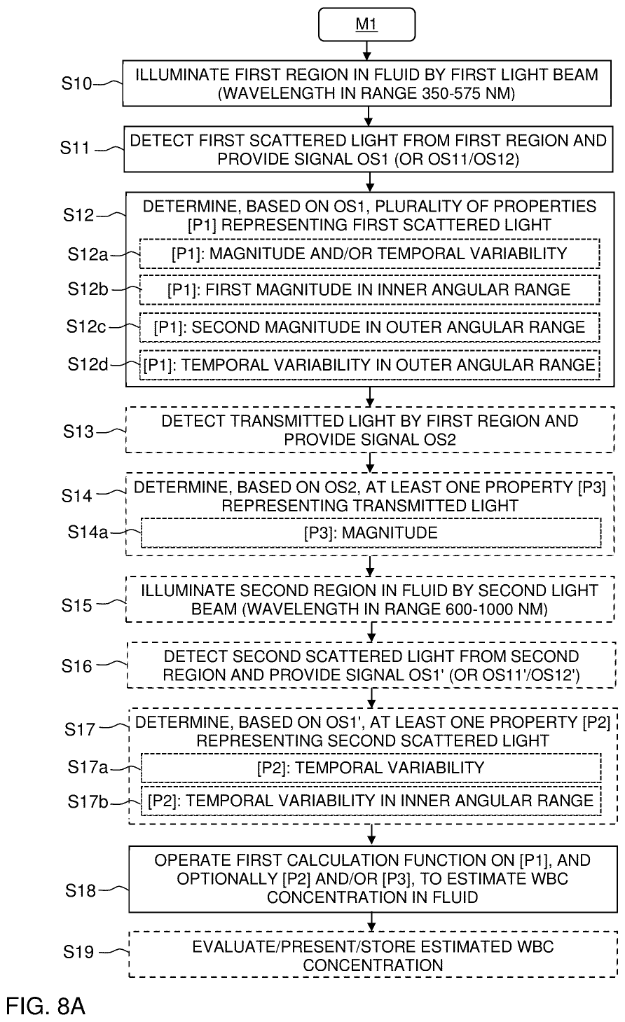

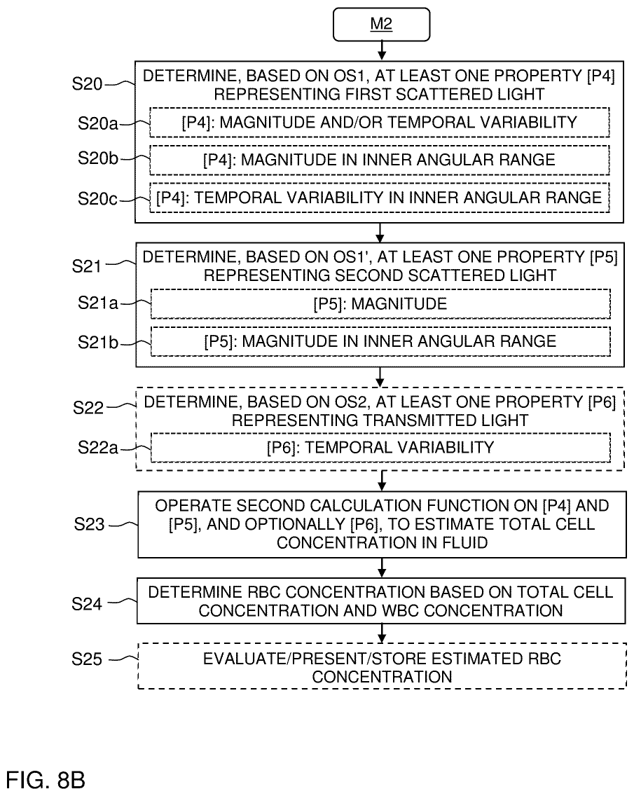

After significant experimentation and testing, the present Applicant has developed a technique detect an optical response to illumination that is specific to WBCs, irrespective of RBCs. The technique may be applied to determine the WBC concentration in effluent from PD therapy, in accordance with a method to be described below with reference to FIG. 8A. The technique enables early detection of peritonitis in patients undergoing PD and reduces the risk for serious complications.

The technique may be extended to estimate the RBC concentration in the effluent, even if the effluent contains WBCs. The extended technique enables early detection of blood in the effluent, allowing the caretaker to investigate the origin of the blood. If the origin is the catheter, an early remedy reduces the risk for complications. If the origin is a serious health issue, the early detection may be lifesaving. The extended technique is described below with reference to FIG. 8B.

To facilitate understanding of the developed technique, a description will first be given of example detection arrangements that have been used by the Applicant when developing the technique. A description is also given of available measurement signals, as well as experimental results on which the technique is at least partly based.

FIG. 1B is a side view, partly in section, of an example measurement device 21 arranged on a tubing portion 17, which may be part of the patient line 15 or the drain line 16. The measurement device 21 includes an illumination system (“light emitting arrangement”) 30 and a detection system (“light detection arrangement”) 31. In the illustrated example, the measurement device 21 also includes a holder 33, which is configured to engage with the tubing portion 17 to achieve a proper alignment of the tubing portion 17 with the illumination system 30 and the detection system 31. In FIG. 1B, the holder 33 comprises first and second walls 37, 38 which define a slot for receiving the tubing portion 17. The spacing between the walls 37, 38 is equal to or smaller than the outer diameter of the tubing portion 17, so that the tubing portion 17 is squeezed, frictionally held or otherwise fixed in the holder 33. At least part of the respective wall 37, 38 is transparent or translucent. In the example of FIG. 1B, the respective wall 37, 38 defines a through-hole or opening 37a, 38a. In some embodiments, transparent/translucent windows may be arranged in the openings 37a, 38a.

The illumination system 30 comprises at least one light source 34, which is configured to emit a light beam 300. The light source 34 may comprise a light-emitting diode (LED) or a miniaturized laser device such as a diode laser. In some embodiments, the illumination system 30 further comprises beam-forming optics 35, which may be arranged to focus the light beam 300 inside the holder 30, for example at a nominal location halfway between the walls 37, 38. Alternatively or additionally, the beam-forming optics 35 may be configured to achieve a predefined transverse beam profile of the light beam 300. The beam-forming optics 35 may comprise one or more lenses. The light emitting device 34 is aligned with the walls 37, 38 so that the light beam 300 passes the openings 37a, 38a. The light beam 300 thereby defines a target volume inside the tubing portion 17. The light source 34 may be configured to generate time-continuous light or pulsed light.

The detection system 31 comprises light-detection devices (“detectors”) 36a, 36b, which are responsive to the emitted light and provide a respective output signal OS1, OS2 that represents the amount of incident light (“intensity”) on the respective detector 36a, 36b. The detector 36a is arranged to detect scattered light and is offset transversely to the light beam 300. The detector 36b is arranged to detect transmitted light and is thus aligned with the light beam 300. In some embodiments (not shown), the detection system 31 comprises detection optics to direct incoming light onto the respective detector 36a, 36b. The output signals OS1, OS2 are time-varying and comprises signal values that represent the momentary amount of incident light at different times. The detectors 36a, 36b may be separate units, such as photodiodes, photoresistors, phototransistors, etc. Alternatively, the detectors 36a, 36b may be formed by different portions of a light-sensing device, for example a photodiode, an array sensor, etc.

As indicated in FIG. 1B, the light beam 300 interacts with particles 303 contained in the effluent within the tubing portion 17. In this interaction, the respective particle 303 may absorb and/or scatter photons in the light beam 300. Scattering is represented by dashed arrows in FIG. 1B.

The effluent may be pumped through the tubing portion 17 at a flow rate F. In some embodiments, the detection system 31 is operated to detect light during a sequence of detection periods, where each detection period results in a signal value in the output signal. The minimum time between starts of detection periods may be set in relation to the expected or actual flow rate of the fluid through the tubing portion 17. A detection period may be achieved by selectively activating the respective detector 36a, 36b to be responsive to light and/or by selectively opening a shutter (not shown) in front of the detector 36a, 36b. Additionally or alternatively, detection periods may be achieved by pulsing the light beam 300.

In a variant, the effluent is stationary in the tubing portion 17 during the measurement.

The use of pulsed light allows for the impact of ambient light on the measurement to be suppressed, if the detection system 31 is operated to detect light during and between light pulses, respectively. Thereby, the light detected between light pulses represents ambient light and may be subtracted from the light detected during light pulses to substantially remove the influence of ambient light. Such subtraction may be performed by the detection system 31 or the computing apparatus 22.

The overall operation of the measurement device 21 is controlled by a control unit 40, which may be configured to generate control signals C1, C2 for the illumination system 30 and the detection system 31. The control signals C1, C2 may control activation of the light source(s) 34 and the detectors 36a, 36b, respectively, as well as any shutter, if present.

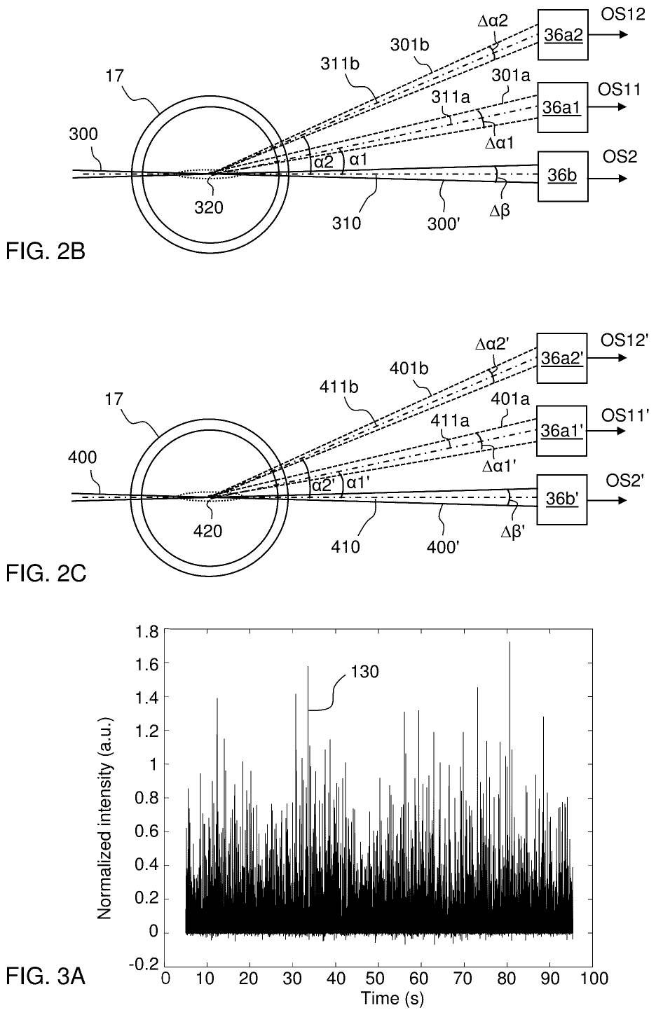

FIG. 2A is a side view, partly in section, of an example arrangement on the detection side of an OPA. Although the tubing portion 17 is shown with a circular cross-section, it could have shape, for example rectangular. If the tubing portion 17 is deformable, the tubing portion may be deformed to define approximately planar and parallel wall portions facing the illumination system 30 and the detection system 31, for example as described in aforesaid WO2022/008213.

As seen in FIG. 2A, the light beam 300 has a main direction 310 towards the tubing portion 17 and defines a target volume 320 within the effluent in the tubing portion 17. The target volume 320 is the volume from which the detection system 31 receives the main portion of the scattered light as the light beam 300 interacts with particles in the effluent. In the illustrated example, the light beam 300 is focused to increase the radiance of the light that interacts with the particles, to thereby increase the amount of scattered light. The detector 36b is aligned with the main direction 310 to receive at least part of the light that passes the effluent (“transmitted light”). The detector 36a is arranged to receive scattered light at a predefined detection angle α to the main direction 310. As used herein, a detection angle given by the angle between the main direction 310 and the center line 311 of the scattered light that reaches the detector 36a, with the detection angle being measured at the center of the target volume 320. Since the light-sensing area of the detector 36a has an extent, the detector 36a will typically receive scattered light within a detection cone 301 (“angular range”) that has an angular width Δα. The angular width of the detection cone 301 may differ in different directions depending on the shape of the light-sensing area. As used herein, the angular width refers to the largest dimension of the detection cone. Generally, the scattered light is symmetrically distributed around the main direction 310. In some embodiments, the detector 36a is ring-shaped to maximize the amount of scattered light that is detected. The width of the ring defines the angular width Δα. For example, the detector 36a may be shaped as a circular ring with uniform width. As shown, the detector 36b may be configured to detect transmitted light within a detection cone 300′ with angular width Δβ. As will be described further below, angular specificity in light detection may serve to improve the accuracy of the concentration values that are calculated by the computing apparatus 22.

The computing apparatus 22 is coupled to the detectors 36a, 36b to receive the output signals (OS1, OS2 in FIG. 1B) and output a result signal RS with concentration values. The concentration values may designate the concentration of WBCs, RBCs or both in the effluent.

The computing apparatus 22 may be implemented by hardware or a combination of software and hardware. In the example of FIG. 2A, the computing apparatus 22 comprises processor circuitry 22a and memory 22b. The processor circuitry 22a may e.g. include one or more of a CPU (“Central Processing Unit”), a DSP (“Digital Signal Processor”), a microprocessor, a microcontroller, an ASIC (“Application-Specific Integrated Circuit”), a combination of discrete analog and/or digital components, or some other programmable logical device, such as an FPGA (“Field Programmable Gate Array”). The memory 22b may include any form of conventional computer memory. The computing apparatus 22 may be at least partly operated in accordance with a control program comprising computer instructions. The control program is stored in the memory 22b and executed by the processor circuitry 22a. The control program may be supplied to the computing apparatus on a computer-readable medium, which may be a tangible (non-transitory) product (e.g. magnetic medium, optical disk, read-only memory, flash memory, etc.) or a propagating signal.

FIG. 2B depicts an alternative configuration in which scattered light is detected at two spatially separate detection angles α1 and α2 by a first detector 36a1 and a second detector 36a2, which generate output signals OS11, OS12. Scattered light is detected by the detector 36a within a first detection cone 301a (“first angular range”) that has an angular width Δα1 and within a second detection cone 301b (“second angular range”) that has an angular width Δα2. The detection angle α1 is defined between the main direction 310 and the center line 311a of the first detection cone 301a. The detection angle α2 is defined between the center line 311b of the second detection cone 301b and the main direction 310. The first detection cone 301a is located closer to the main direction 310 than the second detection cone 301b. Thus, the output signal OS11 represents “inner scattered light”, and the output signal OS12 represents “outer scattered light”.

In FIG. 2B, like in FIG. 2A, transmitted light is received by a detector 36b, which is aligned with the light beam 300 and configured to detect transmitted light within a detection cone 300′ with angular width Δβ. The output signal OS2 of the detector 36b represents the transmitted light.

As will be explained further below, it may be beneficial to illuminate the effluent by light beams at two different wavelengths. The illustration in FIG. 2B may be seen to represent illumination of the effluent by a first light beam 300 at a shorter wavelength located within a first wavelength band. FIG. 2C is identical to FIG. 2B and shows illumination of the effluent by a second light beam 400 at a longer wavelength located within a second wavelength band.

The light beams 300, 400 have a confined spectral width. In some embodiments, and in all experiments presented herein, narrowband light is used for the illumination of the target regions 320, 420. In the context of the present disclosure, narrowband light has a spectral width of less than 20 nm, 10 nm or 5 nm, given as FWHM (full width at half maximum). To generate narrowband light, the light source 24 typically includes a laser, for example a semiconductor-based laser comprising one or more laser diodes.

The Applicant has identified two wavelength bands with differing interaction between light and RBCs, on the one hand, and between light and WBCs, on the other hand. A first (lower) wavelength band extends from about 350 nm to about 575 nm. The first wavelength band may extend further into the ultraviolet (UV), for example to 200 nm, but such shorter wavelengths are currently not believed to applicable for practical use because of a lack of commercially available small-size light sources. Further, the tubing portion 17 may be made of plastics, which typically exhibit significant absorption of UV light. There is also significant light absorption by oxygen molecules below 200 nm. A second (higher) wavelength band extends from about 600 nm to about 1000 nm. The second wavelength band may extend further into the infrared, for example to 1500 nm.

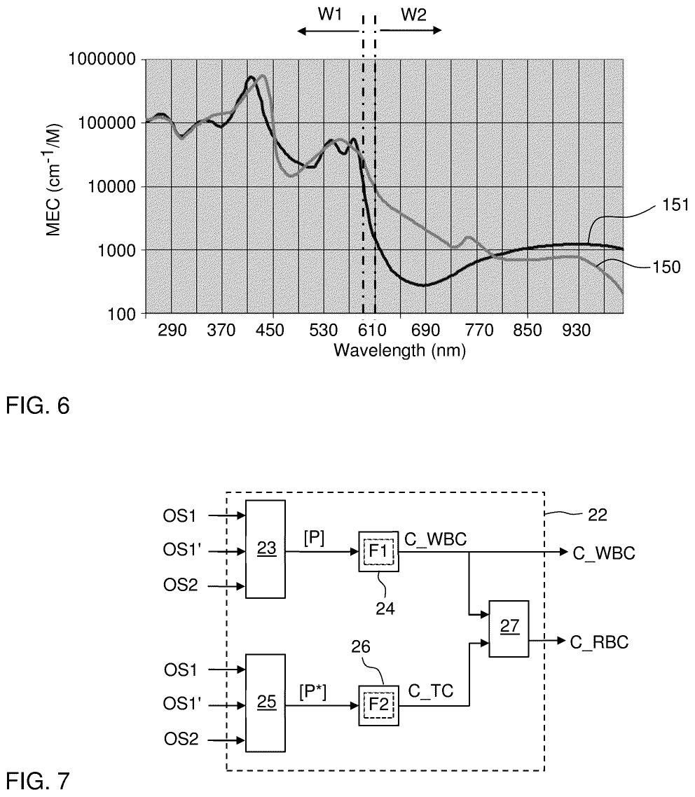

The selection of wavelength bands may be understood based on the absorption spectrum of RBCs. FIG. 6 shows curves 150, 151 of the molar extinction coefficient (MEC) as a function of wavelength for hemoglobin (Hb) and oxyhemoglobin (HbO2), respectively. MEC is a measure of how strongly a chemical species absorbs, and thereby attenuates, light at a given wavelength. Hemoglobin and oxyhemoglobin are carried by RBCs. As seen, absorption is significant at wavelengths below about 600 nm. Above about 600 nm, the absorption decreases rapidly, given than the scale in logarithmic in FIG. 6. Thus, in the first wavelength band W1, the light is primarily absorbed by RBCs, if present, rather than being scattered. Conversely, in the second wavelength band W2, light is primarily scattered by RBCs, if present, rather than being absorbed. For WBCs, the absorption is less dependent on wavelength. Assuming light interacts with WBCs through Mie scattering, scattering will increase with decreasing wavelength. Thus, the interaction of RBCs and WBCs with light will differ between the first and second wavelength bands, which is seen in experiments conducted by the Applicant (below).

As seen in FIG. 6, absorption of RBCs is larger below about 450 nm, with a maximum at 410-430 nm. It may thus be advantageous for the wavelength of the first light beam to be below 450 nm, and in particular in the range of 410-430 nm. The experimental results presented herein are obtained for illuminating light at 405 nm and 650 nm, respectively.

Reverting to FIG. 2C, the second light beam 400 has a main direction 410 towards the tubing portion 17 and defines a target volume 420 within the effluent in the tubing portion 17. Scattered light is detected at two spatially separate detection angles α1′ and α2′ by a first detector 36a1′ and a second detector 36a2′, which generate output signals OS11′, OS12′. Scattered light is detected by the detector 36a′ within a first detection cone 401a (“first angular range”) that has an angular width Δα1′ and within a second detection cone 401b (“second angular range”) that has an angular width Δα2′. The detection angle α1′ is defined between the main direction 410 of the second light beam 400, and the center line 411a of the first detection cone 401a. The detection angle α2 is defined between the center line 411b of the second detection cone 401b and the main direction 410. The first and second detection cones 401a, 401b may be dimensioned in correspondence with the first and second detection cones 301a, 301b. The output signal OS11′ of the detector 36a1′ represents “inner scattered light”, and the output signal OS12′ of the detector 36a2′ represents “outer scattered light”. Transmitted light is received by a detector 36b′, which is aligned with the light beam 400 and configured to detect transmitted light within a detection cone 400′ with angular width Δβ′. The output signal OS2′ of the detector 36b′ thus represents the transmitted light.

FIGS. 2B-2C show the experimental set-up that has been used to explore, develop and refine the technique presented herein. In a practical implementation of the measurement unit 21, the configuration may be different depending on the measurement data that is used for estimating the WBC concentration and/or the RBC concentration. For example, for the first light beam 300 and/or the second light beam 400, a single detection cone may be used, or detection of transmitted light may be omitted. Thus, the number of detectors may be reduced. It is also conceivable to use more than two detection cones for the first light beam 300 and/or the second light beam 400. Likewise, it is conceivable to use more than two light beams at different wavelengths.

It may also be noted that the first and second light beams 300, 400 may illuminate different portions of the effluent in the tubing portion 17. In other words, the target volumes 320, 420 may be spatially separated, for example shifted along the extent of the tubing portion 17. Such a configuration may be simpler to implement, at the cost of more equipment.

In an alternative configuration, the illumination system 30 is configured to direct the first and second light beams to illuminate approximately the same portion of the effluent, but at different time points. This means that the target volumes 320, 420 are very close to each other or effectively overlap. This makes it possible to use the same detectors for detecting scattered and/or transmitted light from the first and second light beams 300, 400.

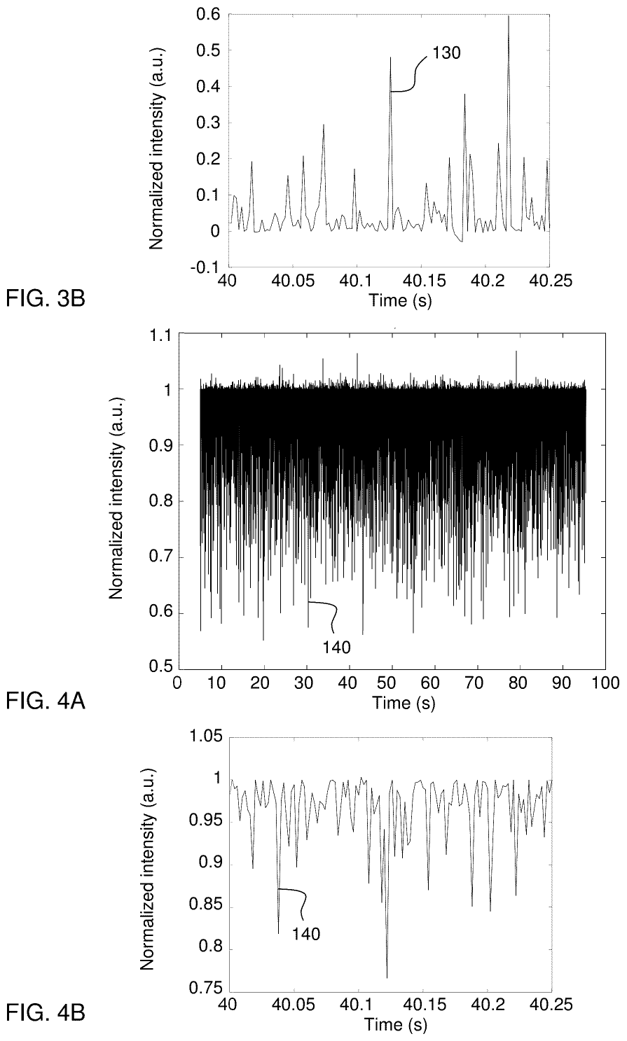

FIG. 3A shows a pre-processed version of an example signal 130 that represents scattered light received by the detector 36a1 in FIG. 2B. The signal 130 is given by the signal OS12 after processing for removal of a baseline by subtraction, to yield normalized intensity. The signal 130 is shown for a time period of 90 seconds. In this example, signal values are provided every 2 ms, which corresponds to the above-mentioned detection period. FIG. 3B is an enlarged view of FIG. 3A and shows individual signal values more clearly.

One reason for the normalization of signals representing scattered light is to reduce the impact of light that originates from scattering of the light beam 300 by the tubing portion 17. The baseline may be given by the lower temporal envelope of the signal OS12. The lower temporal envelope may be determined by any conventional signal processing technique, as readily available to the person skilled in the art, for example by extraction of selected values from the signal OS12 or by operating a Hilbert transformer on the signal OS12. In a non-limiting example, the lower temporal envelope is given by determining the minimum for a sliding window. Alternatively, the baseline may be given by a single value, which is calculated from the signal OS12 and subtracted from all signal values in the signal OS12. In a variant, the baseline is given by the upper temporal envelope of the signal OS2 (below).

FIG. 4A shows a pre-processed version of an example signal 140 that represents transmitted light received by the detector 36b in FIG. 2B. The signal 140 is given by the signal OS2 after processing for removal of a baseline by division, to yield normalized intensity. The signal 140 is given for a time period of 90 seconds, with signal values being provided every 2 ms. FIG. 4B is an enlarged view of FIG. 4A and shows individual signal values more clearly.

One reason for the normalization of signals representing transmitted light is to reduce the impact of changes in the intensity of the light beam 300 over time. The baseline may be given by the upper temporal envelope of the signal OS2. The upper temporal envelope may be determined in correspondence with the lower temporal envelope. In a non-limiting example, the upper temporal envelope is given by determining the maximum for a sliding window. As an alternative or supplement to using a baseline for normalizing the signal OS2, the energy of the laser beam 300 may be measured by a light detector (not shown), for example in the illumination system 30 (FIG. 1B), and used for normalizing the signal OS2, for example by division.

Generally, pre-processing by normalization of the signals from the detectors in the measurement device 21 may improve the accuracy and robustness of the calculated concentration values. However, the data analysis presented further below indicates that acceptable accuracy and robustness is possible without normalization. Further, the data analysis indicates that it may be preferable to use both normalized signals and non-normalized signals in the calculation of concentration values.

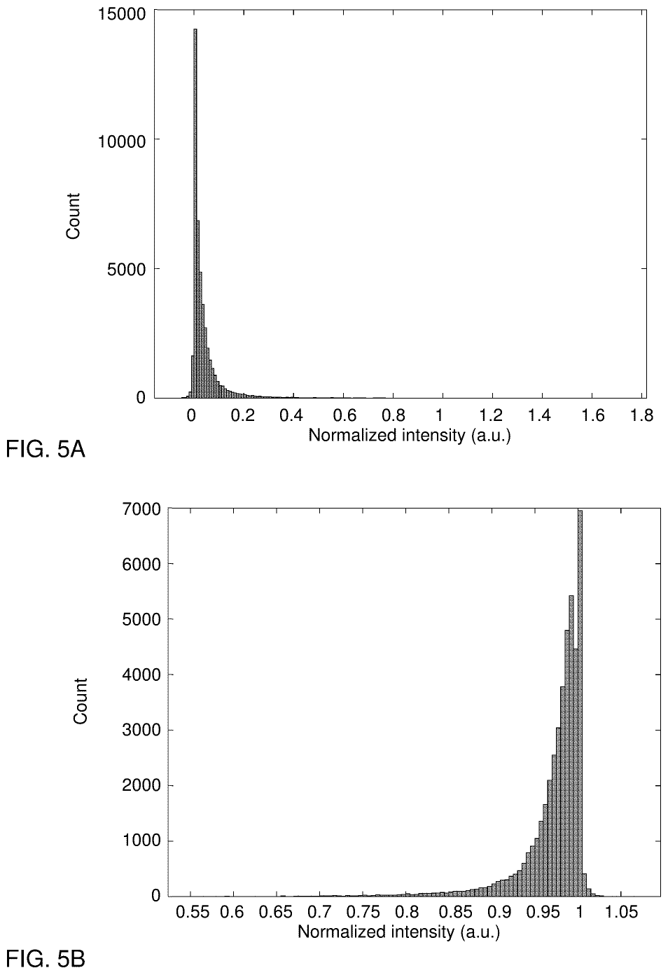

FIG. 5A is a histogram showing the distribution of normalized signal values in the signal 130 in FIG. 3A. Similarly, FIG. 5B is a histogram showing the distribution of normalized signal values in the signal 140 in FIG. 4A. The Applicant has found that, by proper choice of wavelength of the first light beam 300 and the second light beam 400, the distribution of signal values that are acquired during a measurement time period (MTP) changes in dependence of the WBC concentration and/or RBC concentration. This is especially significant for scattered light, but may also be visible in transmitted light.

The MTP may be set to provide a sufficient number of signal values to represent the distribution. In the examples given herein, the distribution is analyzed based on approximately 45 000 signal values, corresponding to MTP being 90 seconds. It is currently believed that the MTP should result in at least 5 000 signal values, and preferably at least 10 000 signal values. Alternatively or additionally, the MTP may be set to be less than about 200, 150, or 100 seconds, and larger than about 5, 10 or 15 seconds. Alternatively or additionally, the MTP may be set in view of the flow rate of the effluent cf. F in FIG. 1B) and/or a desired accuracy of the calculated concentration value(s).

The Applicant has chosen to characterize the distribution of signal values by two main categories: magnitude and variability. Both of these categories are thus estimated based on an ensemble of signal values obtained during the MTP. The variability represents the variation over time (“temporal variability”). In the following, the distribution of normalized signal values is denoted “normalized distribution”, by contrast to the distribution of non-normalized signal values, which is denoted “original distribution”.

For normalized distributions, for example shown in FIGS. 5A-5B, the magnitude may be given by the average or mean of the included signal values (“normalized average”), or by any equivalent measure, such as median or norm. By correlation analysis, the Applicant has found that the k:th percentiles of the original distribution correlates to some degree with the normalized average, at least for k≥5. The term k:th percentile is used in its ordinary meaning to denote a score at or below which a k:th percentage of the signal values falls. The 50:th percentile is equal to the median. Likewise, various quantiles of the original distribution correlate to some degree with the normalized average, including the inter-quartile range (IQR). Further, the average or mean, or any equivalent measure, of the signal values in the original distribution correlates with the normalized average.

For normalized distributions, the variability may be given by the variance of the included signal values (“normalized variability”), or by any equivalent measure, such as any of the variability measures described in aforesaid WO2022/008213, including but not limited to energy, standard deviation, coefficient of variation, variance-to-mean, or Median Absolute Deviation or Mean Absolute Deviation (MAD). By correlation analysis, the Applicant has found that the k:th percentiles of the normalized distribution correlates to some degree with the normalized variability for k≥1. Likewise, various quantiles of the normalized distribution correlate to some degree with the normalized variability, including the inter-quartile range (IQR). Further, the variance, or any equivalent measure, of the signal values in the original distribution correlates with the normalized variability.

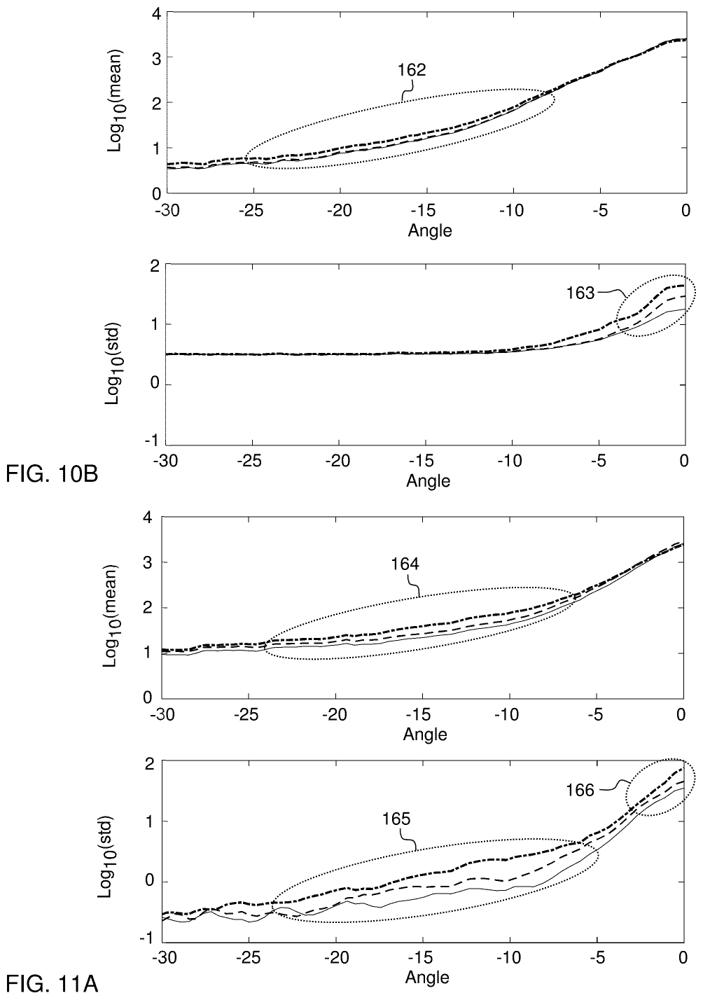

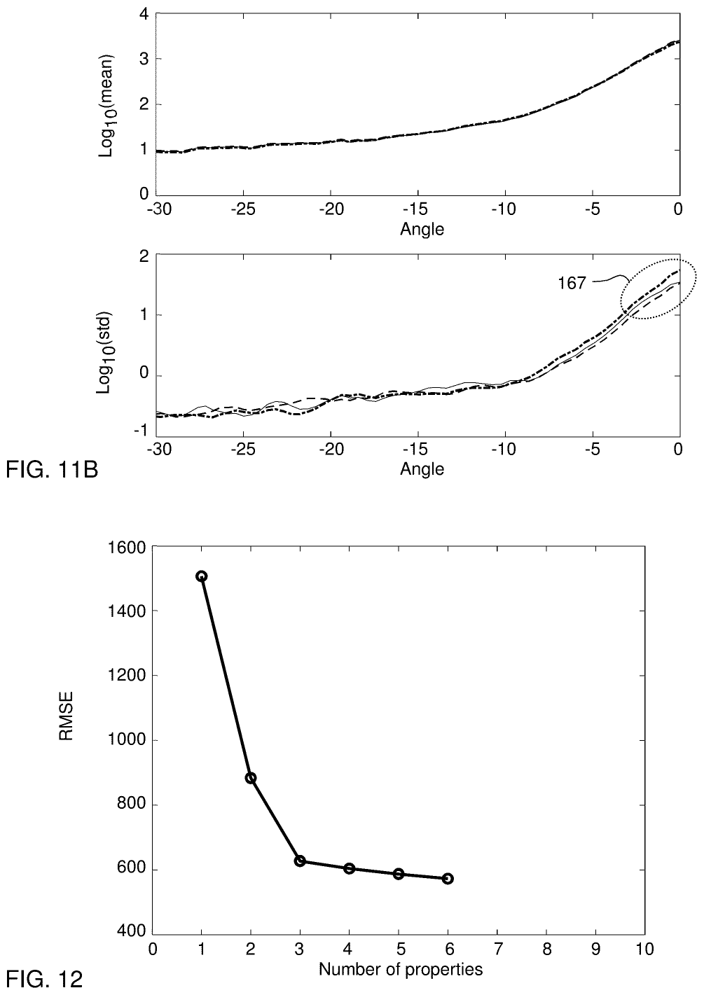

The present Applicant has conducted experiments to analyze the impact of presence of WBCs and RBCs, respectively, on the magnitude and the variability of transmitted and scattered light for a beam of narrowband laser light with a wavelength in the first wavelength band and the second wavelength band, respectively. In these experiments, the detectors 36a1, 36a2, 36b in FIG. 2B and the detectors 36a1′, 36a2′ and 36b′ in FIG. 2C were replaced with a two-dimensional CCD detector, and the light received by the CCD detector was separated by detection angle to the main direction 310. A few examples of the experimental results are shown in FIGS. 10-11.

For the experiments, samples with different concentrations of RBCs were prepared by adding RBCs to phosphate buffered saline (PBS). Similarly, samples with different concentrations of WBCs were prepared by adding WBCs to PBS. FIGS. 10-11 are given for RBC concentrations of 0 cells/μL, 63 cells/μL and 1000 cells/μL, and WBC concentrations of 0 cells/μL, 63 cells/μL and 1000 cells/μL. In FIGS. 10-11, results for concentrations of 0, 63 and 1000 cells/μL are indicated by solid lines, dashed lines and dot-dashed lines, respectively. The results are presented for a laser beam at 405 nm (first wavelength band, W1) and a laser beam at 650 nm (second wavelength band). The laser beam at 405 nm will be referred to as “blue light”, and the laser beam at 650 nm will be referred to as “red light”. Each of FIGS. 10A, 10B, 11A, 11B includes a top graph that shows magnitude as a function of detection angle, and a bottom graph that shows variability as a function of detection angle. The magnitude is given by the average of the detected light at the respective angle within the MTP, and the variability is given by the standard deviation of the detected light at the respective angle within the MTP.

FIG. 10A shows experimental results for illumination of WBC samples with the laser beam at 650 nm (red light). The top graph, in encircled region 160, indicates that the magnitude of scattered light changes with WBC concentration, at least for angles in the range of 7°-30°. The bottom graph, in encircled region 161, indicates that the variability of the transmitted light changes with WBC concentration.

FIG. 10B shows experimental results for illumination of RBC samples with the laser beam at 650 nm (red light). The top graph, in encircled region 162, indicates that the magnitude of scattered light changes with RBC. The response to red light in region is thus similar to the response to The bottom graph, in encircled region 163, indicates that the variability of the transmitted light changes with RBC concentration. For red light, the response to RBCs (regions 162, 163) is thus similar to the response to WBCs (regions 160, 161).

FIG. 11A shows experimental results for illumination of WBC samples with the laser beam at 405 nm (blue light). The top graph, in encircled region 164, indicates that the magnitude of scattered light changes with WBC concentration, at least for angles in the range of 7°-30°. The bottom graph, in encircled region 165, indicates that the variability of scattered light changes with WBC concentration, at least for angles in the range of 7°-30°. The bottom graph further indicates, in encircled region 166, that the variability of transmitted light changes with WBC concentration.

FIG. 11B shows experimental results for illumination of RBC samples with the laser beam at 405 nm (blue light). In the top graph, the magnitude of the detected light is effectively independent of RBC concentration. The bottom graph, in encircled region 167, indicates that the variability of transmitted light changes with RBC concentration.

The results in FIGS. 10-11 indicate that blue light is the best candidate for determining the concentration of WBCs in the presence of RBCs since the both the magnitude and the variability of the scattered light seem to be dependent on WBC concentration (FIG. 11A) and almost independent of, or at least much less dependent, on the RBC concentration (FIG. 11B).

The present Applicant has found that it might be easier to estimate, based on measurement data for scattered and/or absorbed light, the total concentration of particles and then determine the RBC concentration from the total concentration and WBC concentration. In some embodiments, the RBC concentration is given by the difference between the total concentration and the WBC concentration, assuming the contribution to the measurement data from other particles is small. Experiments indicate that the total cell concentration can be determined with relatively high accuracy based on at partly the same signals that are used for determining the WBC concentration.

FIG. 7 is a block diagram of an example computing apparatus 22 in accordance with an embodiment. In the illustrated example, the computing apparatus 22 is configured to receive output signals OS1, OS1′ and OS2 from three detectors in the measurement device 21 (not shown). With reference to FIG. 2A, signal OS1 represents scattered light of wavelength in band W1 (“blue light”) and is obtained from detector 36a, signal OS1′ represents scattered light of wavelength in band W2 (“red light”) and is obtained from a detector not shown in FIG. 2A (cf. detectors 36a1′, 36a2′ in FIG. 2C), and signal OS2 represents transmitted light of wavelength W1 (“blue light”) and is obtained from detector 36b. The computing apparatus 22 comprises a first parameter calculation unit 23, which is configured to calculate values of a first set of predefined parameters (“properties”) based on the signals OS1, OS1′, OS2. Each parameter or property represents one of the above-mentioned categories, namely magnitude or variability, or a combination thereof. The resulting set of property values, [P], is provided to a WBC calculation unit 24, which is configured to estimate the WBC concentration, C_WBC, based on [P], by use of a first calculation function F1. The function F1 defines a predefined relation between C_WBC and [P] and may be given as a mathematical expression or a look-up table.

In the illustrated example, the computing apparatus 22 comprises a second parameter calculation unit 25, which is configured to calculate values of an additional set of predefined parameters (“properties”) based on the signals OS1, OS1′, OS2. Each parameter or property represents one of the above-mentioned categories, namely magnitude or variability, or a combination thereof. The resulting set of property values, [P*], is provided to a TC calculation unit 26, which is configured to estimate the total concentration of particles, C_TC, based on [P*], by use of a second calculation function F2. The function F2 defines a predefined relation between C_TC and [P*] and may be given as a mathematical expression or a look-up table.

The computing apparatus 22 further comprises an RBC calculation unit 27, which is configured to calculate the RBC concentration, C_RBC, based on C_TC and C_WBC. As noted above, C_RBC may be obtained by subtracting C_WBC from C_TC.

In the illustrated example, the computing apparatus 22 is configured to output both C_WBC and C_RBC. With reference to FIG. 2A, C_WBC and C_RBC would be included in the result signal, RS. In a variant, only C_RBC is output. In a further variant, only C_WBC is output. If only C_WBC is calculated and output, units 25-27 are superfluous and may be omitted.

In a further variant, the total concentration of particles, C_TC, is obtained from another measurement device, which may determine C_TC by use of the optical technique described in aforesaid WO2022/008213, turbidimetry, optical coherence tomography (OCT), direct imaging, a Coulter counter, or any other commercially available particle counter.

The computing apparatus 22 may further include a pre-processing unit, which is configured to pre-process one or more of the signals OS1, OS1′, OS2, for example to remove outlier data, to perform the above-mentioned normalization, to perform a high-pass filtration, etc.

The separation into units 23-27 in FIG. 7 is done for illustrative purposes. In practice, the functionality of the computing apparatus 22 may be implemented by any number of units. It is also to be understood that the computing apparatus 22 may be modified to only use a sub-set of the signals OS1, OS1′ and OS2 for the calculations, for example in accordance with the methods in FIGS. 8A-8B.

FIG. 8A is a flowchart of an example method M1 for operating an OPA 20 to determine the concentration of WBCs in a fluid. The fluid may be effluent from an APD cycler. The method M1 will be described with reference to the OPA 20 in FIGS. 1-2. Optional steps are indicated by dashed lines.

In step S10, the illumination system 30 is operated to illuminate the fluid by light in the first wavelength band, W1 (FIG. 6). The illumination system 30 thereby generates the first light beam 300, which is directed to a first region in the fluid. The first region corresponds to the target volume 320 in FIG. 2B. In step S12, the detection system 31 is operated to detect scattered light from the first region and provide an output signal. Here, the scattered light that is detected in step S12 is denoted “first scattered light”. Depending on configuration of the detection system 31, the output signal may be OS1 (FIG. 2A), OS11, OS12, or a combination of OS11 and OS12 (FIG. 2B). Steps S10 and S11 may be performed by the control device 40, shown in FIG. 1B.

In step S12, the computing apparatus 22 obtains the output signal from step S11 and determines, based on this signal, a plurality of first properties, [P1], that represent the first scattered light. In step S18, the computing apparatus 22 operates a first calculation function on [P1] to determine the WBC concentration. The first calculation function corresponds to F1 in FIG. 7.

As used herein, a “property” is a characteristic of light received by a detector and is typically a characteristic of the histogram of signal values within a time window in the output signal from the detector (cf. FIGS. 5A-5B). In other words, the property is determined to represent an ensemble or time series of signal values. The time window corresponds to the above-mentioned measurement time period, MTP. Typically, the property falls into one of two main categories: magnitude or variability. As noted above, both magnitude and variability may be calculated in many different ways. After significant experimentation, the present Applicant has made the surprising finding that the WBC concentration (C_WBC) may be estimated with adequate accuracy based on a plurality of first properties, [P1], for example two first properties. As shown by step S12a, the properties in [P1] may be determined to represent magnitude and/or variability. In some embodiments, [P1] only represents magnitude and thus contains different measures of magnitude for an ensemble of signal values. In some embodiments, [P1] only represents variability and thus contains different measures of variability for an ensemble of signal values. To be useful, these different measures should not be perfectly correlated with each other. It is currently believed that different measures of magnitude are useful if they have a correlation coefficient to each other in the range of 0-0.8. The same applies to different measures of variability. In some embodiments, [P1] includes one or more properties that represent magnitude and one or more properties that represent variability. Generally, the use of both magnitude and variability has been found to improve the accuracy of the estimated C_WBC.

Based on the experiments described with reference to FIGS. 10-11, the Applicant has achieved good results when the first scattered light is received at a detection angle α, which is in a range from about 6° to about 35° and defined in accordance with FIG. 2A. The detection cone of the detector 36a may have any suitable angular width, Aa (FIG. 2A), for example 1°-10°. With reference to FIG. 2A, the detector 36a may be arranged to not receive scattered light at angles outside the range 6°-35° to maximize the relevance of the detected light.

The Applicant has identified a potential for further improvement by through angularly specific detection of the first scattered light. Surprisingly, it has been found that the first scattered light that is received at different detection angles α have different dependencies on the WBC concentration. Thus, in some embodiments, the first scattered light is detected in first and second angular ranges, which differ from each other. The first and second angular ranges corresponds to the detection cones 301a, 301b in FIG. 2B. The first and second angular ranges may partially overlap. However, to maximize the collected information about WBCs, it is believed that the first and second angular ranges should be non-overlapping. Overlapping or not, one angular range is located closer to the main direction 301 than the other. In the following, the first and second angular ranges are therefore denoted “inner angular range”, and “outer angular range”, respectively. In FIG. 2B, the inner angular range corresponds to detection cone 301a, and the outer angular range corresponds to detection cone 301b. The Applicant achieved good results with the inner angular range 301a being fully located within 7°-16° to the main direction 310, and the outer angular range 301b being fully located within 16-35° to the main direction 310. In the notation of FIG. 2B, the inner angular range 301a fulfils:

$$α1 - Δα1 / 2 \geq 7 °$$

$$α1 + Δα1 / 2 \leq 16 °$$

and the outer angular range 301b fulfils:

$$α2 - Δα2 / 2 \geq 16 °$$

$$α2 + Δα2 / 2 \leq 35 °$$

Returning to FIG. 8A, the method M1 may include a step S12b of determining a first magnitude of the first scattered light in the inner angular range 301a, and a step S12c of determining a second magnitude of the first scattered light in the outer angular range 301b. Data analysis indicates that the accuracy of step S18 may be improved if [P1] represents the first and second magnitudes from steps S12b-S12c.

Further analysis of the experimental results indicates that it may be beneficial to include a property in [P1] that represents the variability of the first scattered light detected in the outer angular range 301b. This property may or may not be combined with the first and/or second magnitudes from steps S12b-S12c, or a magnitude determined by step S12a. Thus, the method M1 may include a step S12d of determining the temporal variability of the first scattered light in the outer angular range 301b.

Experiments also indicate that the accuracy of step S18 may be improved by steps S15-S17. In step S15, the illumination system 30 is operated to illuminate the fluid by light in the second wavelength band, W2 (FIG. 6). The illumination system 30 thereby generates the second light beam 400, which is directed to a second region in the fluid. The second region corresponds to the target volume 420 in FIG. 2C. In step S16, the detection system 31 is operated to detect scattered light from the second region and provide an output signal. Here, the scattered light that is detected in step S16 is denoted “second scattered light”. Depending on configuration of the detection system 31, the output signal may be a signal OS1′ corresponding to OS1 in FIG. 2A. Alternatively, the output signal may be OS11′, OS12′, or a combination of OS11′ and OS12′ in FIG. 2C. Steps S15 and S16 may be performed by the control device 40, shown in FIG. 1B.

In step S17, the computing apparatus 22 obtains the output signal from step S16 and determines, based on this signal, at least one second property, [P2], that represents the second scattered light. In step S18, the computing apparatus 22 operates the first calculation function on [P1] and [P2] to determine the WBC concentration. Data analysis indicates an improvement when [P2] includes a property that represents the variability of the second scattered light. Thus, the method M1 may include a step S17a of determining the temporal variability of the second scattered light. It is currently believed, backed by experimental data, to be beneficial if [P2] includes the variability of the second scattered light in the inner angular range. Thus, step S17a may be replaced by a step S17b of determining the temporal variability of the second scattered light in the inner angular range.

Experiments also indicate that the accuracy of step S18 may be improved by steps S13-S14. In step S13, the detection system 31 is operated to detect transmitted light from the first region and provide an output signal. Step S13 is performed based on the first light beam (in the first wavelength band, W1) that is generated by step S10. Step S13 may or may not be performed concurrently with step S11. The output signal corresponds to OS2 in FIGS. 2A-2B. Step S13 may be performed by the control device 40, shown in FIG. 1B.

Based on the experiments described with reference to FIGS. 10-11, the Applicant has found that good results are achieved when the transmitted light is detected within a detection cone that has a width Δβ (FIGS. 2A-2B) of less than 6° and preferably less than 5° or 4°, to limit the impact of the first scattered light on the measurement.

In step S14, the computing apparatus 22 obtains the output signal from step S13 and determines, based on this signal, at least one third property, [P3], that represents the transmitted light. In step S18, the computing apparatus 22 operates the first calculation function on [P1] and [P3] to determine the WBC concentration. Data analysis indicates an improvement when [P3] includes a property that represents the magnitude of the transmitted light. Thus, the method M1 may include a step S14a of determining the magnitude of the transmitted light.

Data analysis also indicates that it may be beneficial to operate the first calculation function on [P1], [P2] and [P3] to determine the WBC concentration in step S18.

Data analysis further indicates that the estimate of the WBC concentration may be improved by using, in step S18, at least one additional property [P′] that represents the transmitted light from the second region. Thus, although not shown in FIG. 8A, the method M1 may include a step of operating the detection system 31 to detect transmitted light from the second region and provide an output signal. This step is performed based on the second light beam (in the second wavelength band, W2) that is generated by step S15. The output signal corresponds to OS2′ in FIG. 2C. Data analysis indicates a potential improvement when [P′] represents the variability of the transmitted light. Thus, the method M1 may include a step of determining the variability of the transmitted light from the second region. In step S18, [P′] may replace or supplement [P2] or [P3].

As shown by step S19, the method M1 may involve an evaluation of the WBC concentration from step S18, for example for detection of potential peritonitis. The evaluation may comprise comparing the WBC concentration to a threshold value. If an elevated WBC concentration is detected, the caretaker may be alerted thereof. The evaluation may be performed by the control system of the cycler 11a (FIG. 1A), and the caretaker may be alerted through a feedback unit. The feedback unit may comprise one or more of a display, an indicator lamp, a speaker, a buzzer, a beeper, a vibrator, etc. For example, the feedback unit may display one or more of the WBC concentration, the WBC concentration compared to the threshold value, a percentage of the WBC concentration compared to the threshold value, and a textual and/or graphical indication that the WBC concentration is indicative of potential or early peritonitis. Additionally, the WBC concentration and one or more metrics derived thereof may also be stored over time and then displayed such that one may be able to see how the WBC concentration and one or more metrics derived thereof for a patient have changed over time such as, e.g., over days, weeks, or months of peritoneal dialysis. For example, a graph and/or table of the WBC concentration and one or more metrics derived thereof over time may be displayed. Further, the feedback unit may be included in the cycler 11a, the OPA 20, or in a separate device. Alternatively or additionally, step S19 may involve presenting the WBC concentration to the caretaker through the feedback device. Alternatively or additionally, step S19 may involve storing the WBC concentration for the patient, for example on a memory of the cycler 11a or on a remote storage device, such as a server or a cloud service.

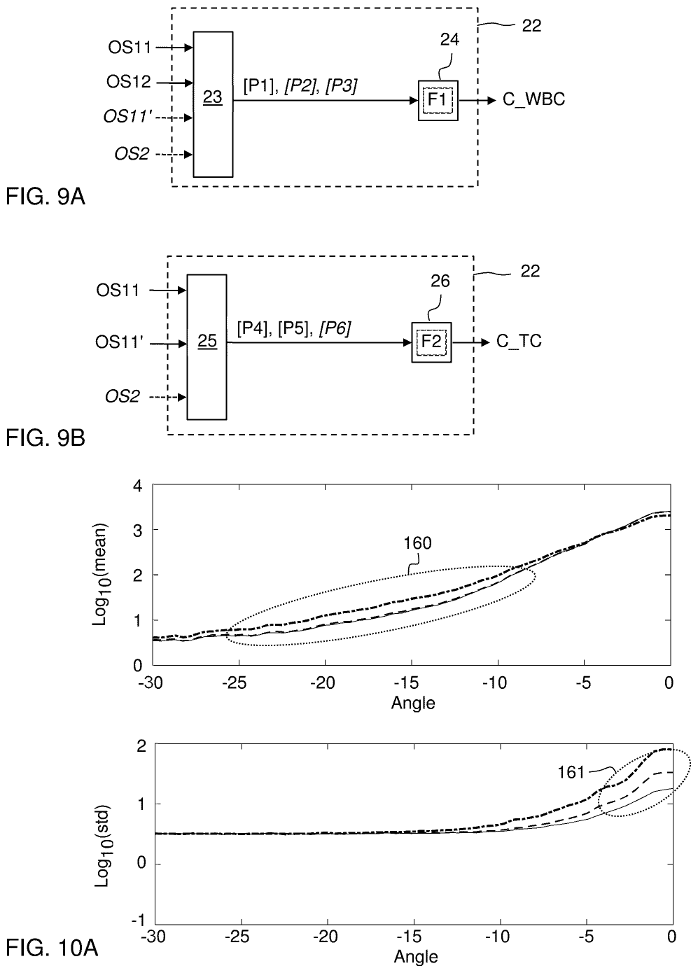

FIG. 9A is a block diagram of an example computing apparatus 22 which is configured to implement embodiments of the method M1 of FIG. 8A. The first parameter calculation unit 23 is configured to receive at least the signals OS11 and OS12, which represent the scattered light of the first light beam 300 in the inner and outer angular ranges 301a, 301b (FIG. 2B). As indicated by dashed arrows, the unit 23 may optionally be configured to receive the signal OS11′, which represents the second scattered light in the inner angular range 401a (FIG. 2C), and/or the signal OS2, which represents the transmitted light of the first light beam 300 (FIG. 2B). The unit 23 is configured to determine the plurality of first properties, [P1], based on OS11 and OS12, in accordance with step S12 (FIG. 8A). As indicated by italic characters, the unit 23 may optionally be configured to determine the at least one second property, [P2], based on OS11′, in accordance with step S18. Alternatively or additionally, the unit 23 may be configured to determine the at least one third property, [P3], based on OS2, in accordance with step S14. The WBC calculation unit 24 is configured to determine C_WBC by operating the first calculation function, F1, on [P1], optionally in combination with [P2] and/or [P3], in accordance with step S17. The computing apparatus 22 is configured to output C_WBC for further processing, for example in accordance with step S19.

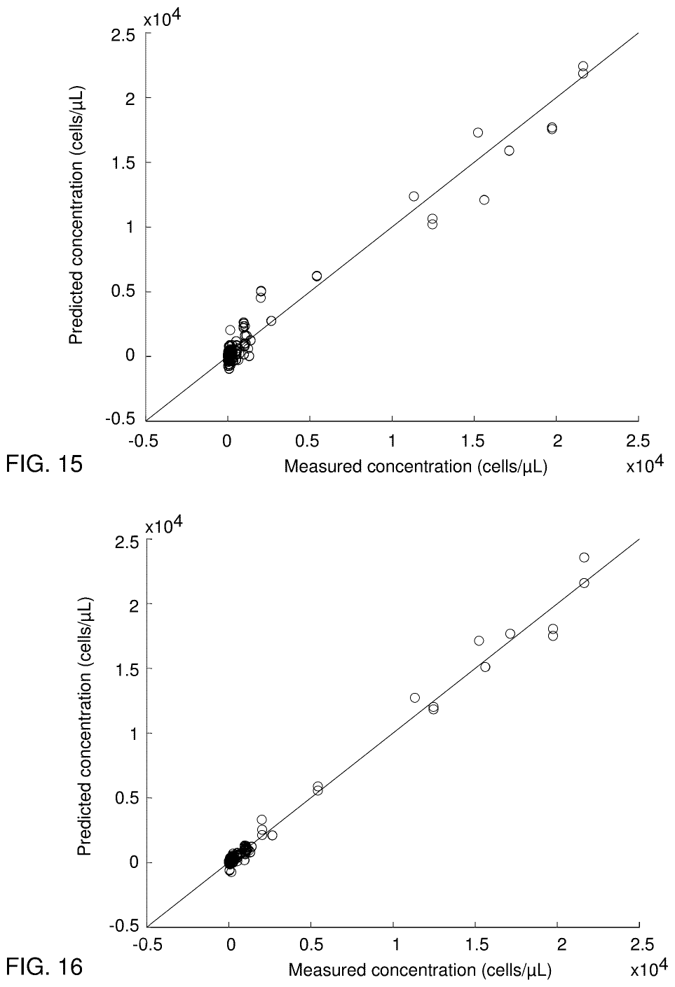

The number of properties that are extracted and used in the first and second calculation functions F1, F2 has been found to influence the accuracy of the calculated concentrations. FIG. 12 is a graph of prediction error (RMSE) as a function of the number of properties included in the second calculation function for a linear dependence on the properties (cf. Equation 3, below). The prediction error is determined based on the experiments and results described below with reference to FIGS. 15-16. As seen, the prediction error decreases significantly when going from one to two properties, which is believed to be the minimum number of properties to be used. It is also seen that the prediction error levels out when more than three properties are included. Thus, based on FIG. 12, it seems as if using four or more properties does not add much in terms of prediction accuracy of the second calculation function. The same dependence of prediction error on the number of properties is seen for the first calculation function.

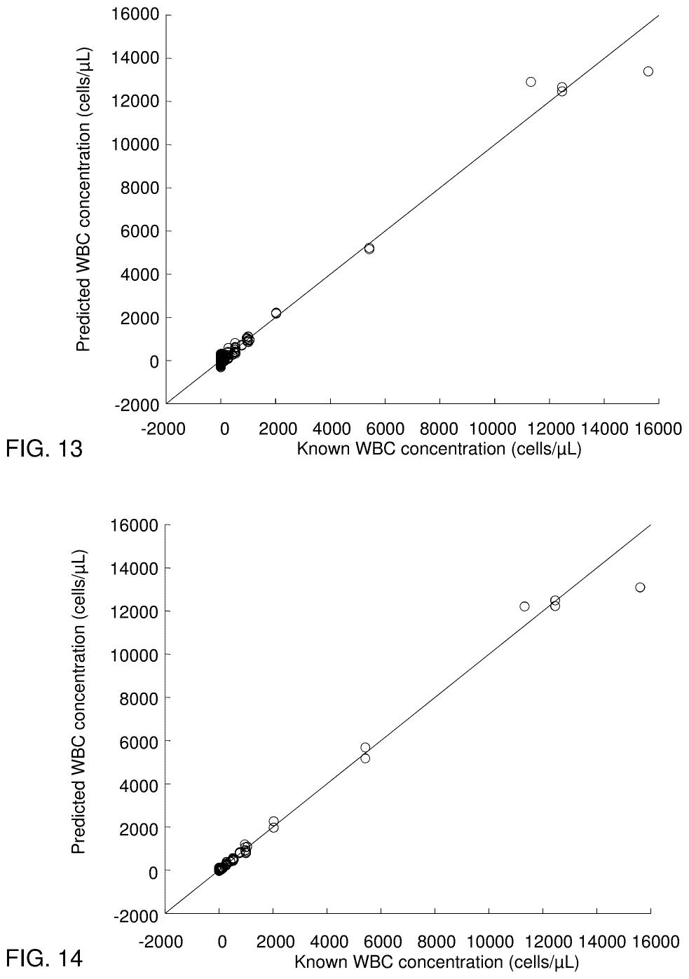

Experiments have been performed to determine the first calculation function, F1. In these experiments, reference fluids were prepared with known concentrations of WBCs and RBCs. Specifically, the reference fluids had WBC concentrations in the range of 0-15000 cells/μL, and RBCs were included at concentration ratios of 0, 0.2, 0.5 and 0.8. The method M1 was performed for each of the reference fluids for different combinations of extracted properties, resulting in predicted values of WBC concentration. Some example results are presented in FIGS. 13-14.

FIG. 13 is a graph of predicted WBC concentration versus known WBC concentration for a linear dependence on three specific properties. The solid line represents a one-to-one relation between predicted and known WBC concentrations. The high degree of correlation shows that WBC concentration in a fluid can be determined by use of the method M1 irrespective of RBC concentration in the fluid. In the example of FIG. 13, the predicted WBC concentration is determined based on [P1] only, given by step S12 in FIG. 8A. In the illustrated example, [P1] consists of a property Pla representing the magnitude of first scattered light in the outer angular range 301b (FIG. 2B), a property P1b representing the variability of first scattered light in the outer angular range 301b, and a property Plc representing the magnitude of first scattered light in the inner angular range 301a. A linear correlation was used, represented as:

$$y = a + b \cdot P 1 a + c \cdot P 1 b + d \cdot P 1 c \qquad ( 1 )$$

with y being the predicted WBC concentration, and a-d being weights or coefficients given by the linear regression. The first calculation function F1, used by step S18, may be given by Equation (1).

In the specific example of FIG. 13, Pla is given by the 90:th percentile of the signal values within a time window (MTP=90 seconds) in the signal OS12 (FIG. 2A), P1b is given by the 75:th percentile of the signal values within the MTP in the signal OS12 after normalization (by subtraction of baseline), and Plc is given by the 90:th percentile of the signal values within the MTP in the signal OS11. The correlation of data in FIG. 13 yields R=0.985, MAE=143.3 and RMSE=268.7, with R being the correlation coefficient, MAE being the mean absolute error, and RMSE being the root means square error.

FIG. 14 is a graph of predicted WBC concentration versus known WBC concentration when including a second order dependence on specific properties. FIG. 14 shows that WBC concentration in a fluid can be determined irrespective of RBC concentration. In the example of FIG. 14, the predicted WBC concentration is determined based on [P1] and [P2], given by steps S12 and S17 in FIG. 8A. In the illustrated example, [P1] consists of a property Pla representing the magnitude of first scattered light in the outer angular range 301b (FIG. 2B), a property P1b representing the variability of first scattered light in the outer angular range 301b, and a property P2a representing the variability of second scattered light in the inner angular range 401a (FIG. 2C). A linear correlation was used, represented as:

$$y = a + b \cdot P 1 a + c \cdot P 1 b + d \cdot P 2 a + e \cdot P 1 a \cdot P 1 b + f \cdot P 1 a \cdot P 2 a + g \cdot P 1 b \cdot P 2 a + h \cdot {( P 1 a )}^{2} + i \cdot {( P 1 b )}^{2} + j \cdot {( P 2 a )}^{2} \qquad ( 2 )$$

with y being the predicted WBC concentration, and a-j being weights or coefficients given by the linear regression. The first calculation function F1, used by step S18, may be given by Equation (2). If one or more of the coefficients a-j is small, it may be set to zero (0). For example, in the analysis underlying FIG. 14, coefficients d and j are about 1/1000 and 1/100, respectively, of the size of the other coefficients.

In the specific example of FIG. 14, Pla is given by the 90:th percentile of the signal values within a time window (MTP=90 seconds) in the signal OS12 (FIG. 2B), P1b is given by the 75:th percentile of the signal values within the MTP in the signal OS12 after normalization (by subtraction of baseline), and P2a is given by the MAD of the signal values within the MTP in the signal OS11 after normalization (by subtraction of baseline). The correlation of data in FIG. 14 yields R=0.989, MAE=62.9 and RMSE=232.1.

It should be noted that the extracted properties used in in Equations 1 and 2 are merely given as non-limiting examples.

FIG. 8B is a flowchart of an example method M2 for operating an OPA 20 to determine the concentration of RBCs in a fluid. The method M2 will be described with reference to the OPA 20 in FIGS. 1-2. Optional steps are indicated by dashed lines. Steps S20-S24 are performed by the computing apparatus 22 and presumes that at least steps S10-S11 and S15-S16 of the method M1 have been performed.

In step S20, the computing apparatus 22 obtains the output signal from step S11 (FIG. 8A) and determines, based on this signal, at least one fourth property, [P4], that represents the first scattered light. As shown by step S20a, [P4] may be determined to represent magnitude and/or variability. As shown, the method M2 may include a step S20b of determining a property for [P4] that represents the magnitude of the first scattered light in the inner angular range 301a, and/or a step S20c of determining a property for [P4] that represents the variability of the first scattered light in the inner angular range 301a. Data analysis indicates that the accuracy of step S20 may be improved if [P4] represents at least one of the properties from steps S20b-S20c.

In step S21, the computing apparatus 22 obtains the output signal from step S16 (FIG. 8A) and determines, based on this signal, at least one fifth property, [P5], that represents the second scattered light. Data analysis indicates that the accuracy of step S21 may be improved if [P5] represents magnitude. Thus, step S21 may include a step S21a of determining the magnitude of the second scattered light. It is currently believed, backed by experimental data, to be beneficial if [P5] includes the magnitude of the second scattered light in the inner angular range. Thus, step S21a may be replaced by a step S21b of determining the magnitude of the second scattered light in the inner angular range.

In step S23, the computing apparatus 22 operates a second calculation function on [P4] and [P5] to determine the TC concentration. The second calculation function corresponds to F2 in FIG. 7.

In step S24, the computing apparatus 22 determines the RBC concentration based on the TC concentration from step S23 and the WBC concentration from step S18, for example by subtracting the WBC concentration from the TC concentration.

As shown by step S25, the method M2 may involve an evaluation of the RBC concentration from step S24, for example for detection of a risk for complications with the catheter or illness of the patient. The evaluation may comprise comparing the RBC concentration to a threshold value and, if deemed necessary, alert the caretaker. The evaluation may be performed by analogy with step S19. Alternatively or additionally, step S25 may involve presenting the RBC concentration to the caretaker. Alternatively or additionally, step S25 may involve storing the RBC concentration for the patient.

Experiments indicate that the accuracy of step S23 may be improved by step S22.

In step S22, the computing apparatus 22 obtains the output signal from step S14 and determines, based on this signal, at least one sixth property, [P6], that represents the transmitted light of the first light beam. In step 23, the computing apparatus 22 operates the second calculation function on [P4], [P5] and [P6] to determine the TC concentration. Data analysis indicates an improvement when [P6] includes a property that represents the variability of the transmitted light. Thus, step S22 may include a step S22a of determining the temporal variability of the transmitted light of the first light beam.

FIG. 9B is a block diagram of an example computing apparatus 22 which is configured to implement embodiments of the method M2 of FIG. 8B. The second parameter calculation unit 25 is configured to receive at least the signals OS11 and OS11′, which represent the scattered light of the first light beam 300 in the inner angular ranges 301a (FIG. 2B), and the scattered light of the second light beam 400 in the inner angular range 401a (FIG. 2C). As indicated by dashed arrows, the unit 25 may optionally be configured to receive the signal OS2, which represents the transmitted light of the first light beam 300 (FIG. 2B). The unit 25 is configured to determine at least one fourth property, [P4], based on OS11, in accordance with step S20 (FIG. 8B), and at least one fifth property [P5], based on OS11′, in accordance with step S22. As indicated by italic characters, the unit 25 may optionally be configured to determine at least one sixth property, [P6], based on OS2, in accordance with step S22. The RBC calculation unit 26 is configured to determine C_TC by operating the second calculation function, F2, on [P4] and [P5], optionally in combination with [P6], in accordance with step S24. The computing apparatus 22 is configured to output C_TC for further processing, for example in accordance with step S25.

Experiments have been performed to determine the second calculation function F2. The same reference fluids were used as in the experiments presented with reference to FIGS. 13-14. The method M2 was performed for each of the reference fluids for different combinations of extracted properties, resulting in predicted values of TC concentration. Some example results are presented in FIGS. 15-16.

FIG. 15 is a graph of predicted TC concentration versus known TC concentration for a linear dependence on three specific properties. The solid line represents a one-to-one relation between predicted and known TC concentrations. The correlation shows that the TC concentration in a fluid can be determined by use of the method M2. In the example of FIG. 15, the predicted TC concentration is determined based on [P4], [P5] and [P6], given by steps S20, S21 and S22 in FIG. 8B. In the illustrated example, [P4] consists of a property P4a representing the magnitude of first scattered light in the inner angular range 301a, [P5] consists of a property P5a representing the magnitude of second scattered light in the inner angular range 301a, and [P6] consists of a property P6a representing the variability of transmitted light of the first light beam. A linear correlation was used, represented as:

$${y}^{′} = {a}^{′} + {b}^{′} \cdot P 4 a + {c}^{′} \cdot P 4 b + {d}^{′} \cdot P 5 a \qquad ( 3 )$$

with y′ being the predicted TC concentration, and a′-d′ being weights or coefficients given by the linear regression. The second calculation function F2, used by step S23, may be given by Equation (3).

In the specific example of FIG. 15, P4a is given by the 5:th percentile of the signal values within a time window (MTP=90 seconds) in the signal OS11 (FIG. 2B), P5a is given by the 25:th percentile of the signal values within the MTP in the signal OS11′, and Poa is given by the IQR of the signal values within the MTP in the signal OS2 after normalization (division by baseline). The correlation of data in FIG. 15 yields R=0.959, MAE=595.1 and RMSE=873.3.

FIG. 16 is a graph of predicted TC concentration versus known TC concentration when including a second order dependence on specific properties. FIG. 16 is based on the same properties P4a, P5a and Poa as FIG. 15. A linear correlation was used, represented as:

$${y}^{′} = {a}^{′} + {b}^{′} \cdot P 4 a + {c}^{′} \cdot P 5 a + {d}^{′} \cdot P 6 a + {e}^{′} \cdot P 4 a \cdot P 5 a + {f}^{′} \cdot P 4 a \cdot P 6 a ++ {g}^{′} \cdot P 5 a \cdot P 6 a + {h}^{′} \cdot {( P 4 a )}^{2} + {i}^{′} \cdot {( P 5 a )}^{2} + {j}^{′} \cdot {( P 6 a )}^{2} \qquad ( 4 )$$

with y′ being the predicted TC concentration, and a′-j′ being weights or coefficients given by the linear regression. The second calculation function F2, used by step S23, may be given by Equation (4). If one or more of the coefficients a′-j′ is small, it may be set to zero (0).

The correlation of data in FIG. 16 yields R=0.990, MAE=440.9 and RMSE=253.4.

It should be noted that the extracted properties used in Equations 3 and 4 are merely given as non-limiting examples. To give a further non-limiting example, a result comparable to the one in FIG. 15 may be obtained by using only [P4] and [P5] to determine the predicted TC concentration. For example, this may be achieved when [P4] comprises the above-mentioned properties P4a and P5a, and a property P4b that represents the variability of first scattered light in the inner angular range. In a specific example, P4a is given by the median of the signal values within the MTP in the signal OS11 (FIG. 2B), P4b is given by the 1:st percentile of the signal values within the MTP in the signal OS11 after normalization (by subtraction of baseline), and P5a is given by the 25:th percentile of the signal values within the MTP in the signal OS11′ (FIG. 2C).

The available data indicates that calculation functions that have a second order dependence on extracted properties may give a higher accuracy of the predicted concentration compared to calculation functions that have a linear dependence on extracted properties. This is particularly noticeable for the second calculation function.

It is conceivable to use calculation functions that have a third or higher order dependence on extracted properties, or another type of non-linear dependence.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the scope of the appended claims.

For example, the techniques described in the foregoing are not limited to APD but are equally applicable to other types of PD therapy such as CAPD. The techniques are not limited to PD effluent but are equally applicable to other medical fluids that may contain WBCs and RBCs.

Further, while operations are depicted in the drawings in a particular order, this should not be understood as requiring that such operations be performed in the particular order shown or in sequential order, or that all illustrated operations be performed, to achieve desirable results.

In the following, clauses are recited to summarize some aspects and embodiments as disclosed in the foregoing.

C1. An optical detection apparatus for determining a concentration of white blood cells in a fluid, said apparatus comprising: a light emitting arrangement (30), which is configured to generate a first light beam with a wavelength within a range of 350-575 nm and arranged to direct the first light beam (300) along a first main direction (310) into a first region (320) in the fluid; a light detection arrangement (31), which is arranged to detect first scattered light originating from the first light beam (300) in the first region (320) and provide a first signal (OS1; OS11, OS12) that represents the first scattered light as a function of time; and a computing apparatus (22), which is configured to: determine, based on the first signal (OS1; OS11, OS12), a plurality of first properties ([P1]) representing the first scattered light; and operate a first calculation function (F1) on the plurality of first properties ([P1]) to estimate the concentration of white blood cells in the fluid.

C2. The apparatus of C1, wherein the computing apparatus (22) is configured to estimate the concentration of white blood cells in the presence of red blood cells within the fluid.

C3. The apparatus of C1 or C2, wherein the light detection arrangement (31) is arranged to detect the first scattered light at an angle to the first main direction (310) of the first light beam (300) through the first region (320), said angle being in a range extending from about 6° to about 35°.

C4. The apparatus of any preceding clause, wherein the computing apparatus (22) is configured to determine a respective first property to represent an ensemble of signal values within a respective time window of the first signal (OS1; OS11, OS12).

C5. The apparatus of any preceding clause, wherein the plurality of first properties ([P1]) comprises a magnitude of the first scattered light.

C6. The apparatus of any preceding clause, wherein the plurality of first properties ([P1]) comprises a temporal variability of the first scattered light.

C7. The apparatus of any preceding clause, wherein the light detection arrangement (31) is configured to detect the first scattered light in a first angular range (301a) and a second angular range (301b), wherein the first angular range (301a) differs from the second angular detection range (301b), and wherein the plurality of first properties ([P1]) comprise a first magnitude of the first scattered light within the first angular range (301a), and a second magnitude of the first scattered light within the second angular range (301b).

C8. The apparatus of C7, wherein the first angular range (301a) is non-overlapping with the second angular range (301b).

C9. The apparatus of C7 or C8, wherein the first angular range (301a) is closer to the first main direction (310) than the second angular range (301b).

C10. The apparatus of any one of C7-C9, wherein the first angular range (301a) is located within 7°−16° to the first main direction (310), and the second angular range (301b) is located within 16°-35° to the first main direction (310).

C11. The apparatus of C6 in combination with any one of C7-C10, wherein computing apparatus (22) is configured to determine the temporal variability of the first scattered light for the second angular range (301b).

C12. The apparatus of any preceding clause, wherein the light emitting arrangement (30) is configured to generate a second light beam (400) with a wavelength within a range of 600-1000 nm and is arranged to direct the second light beam (400) along a second main direction (410) into a second region (420) in the fluid, wherein the light detection arrangement (31) is arranged to detect second scattered light originating from the second light beam (400) in the second region (420) and provide a second signal (OS1′) that represents the second scattered light as a function of time, and wherein the computing apparatus (22) is configured to determine, based on the second signal (OS1′), at least one second property ([P2]) representing the second scattered light, and operate the first calculation function (F1) on the plurality of first properties ([P1]) and the at least one second property ([P2]) to estimate the concentration of white blood cells.

C13. The apparatus of C12, wherein the computing apparatus (22) is configured to determine the at least one second property ([P2]) to represent an ensemble of signal values within a second time window of the second signal (OS1′).

C14. The apparatus of C12 or C13, wherein the at least one second property ([P2]) comprises a temporal variability of the second scattered light.

C15. The apparatus of C14, wherein the light detection arrangement (31) is configured to detect the second scattered light in a third angular range (401a), which is located within 7°−16° to the second main direction (410), and wherein the computing apparatus (22) is configured to determine the temporal variability of the second scattered light for the third angular range (401a).

C16. The apparatus of any preceding clause, wherein the light detection arrangement (31) is further configured to detect transmitted light of the first light beam (300) by the first region (320) and output a third signal (OS2) representing the transmitted light, and wherein the computing apparatus (22) is configured to determine, based on the third signal (OS2), at least one third property ([P3]) representing the transmitted light, and operate the first calculation function (F1) on the plurality of first properties ([P1]) and the at least one third property ([P3]) to estimate the concentration of white blood cells in the fluid.

C17. The apparatus of C16, wherein the at least one third property ([P3]) comprises a magnitude of the transmitted light.

C18. The apparatus of C16 or C17, wherein the light detection arrangement (31) is arranged to detect the transmitted light within an angular range extending to less than 4° from the first main direction (310).

C19. The apparatus of any preceding clause, wherein the computing apparatus (22) is further configured to estimate a total particle concentration in the fluid, and estimate a concentration of red blood cells in the fluid as a function of the total particle concentration and the concentration of white blood cells.

C20. The apparatus of any one of C12-C15, wherein the computing apparatus (22), to estimate a total particle concentration in the fluid, is further configured to: determine, based on the first signal (OS1; OS11, OS12), at least one fourth property ([P4]) representing the first scattered light; determine, based on the second signal (OS2′), at least one fifth property ([P5]) representing the second scattered light; and operate a second calculation function (F2) on the at least one fourth property ([P4]) and the at least one fifth property ([P5]) to estimate the total particle concentration in the fluid.

C21. The apparatus of C20, wherein the at least one fourth property ([P4]) comprises a magnitude of the first scattered light.

C22. The apparatus of C21 in combination with any one of C7-C11, wherein the computing apparatus (22) is configured to determine the magnitude of the first scattered light for the first angular range (301a).

C23. The apparatus of any one of C20-C22, wherein the at least one fourth property ([P4]) comprises a temporal variability of the first scattered light.

C24. The apparatus of C23 in combination with any one of C7-C11, wherein the computing apparatus (22) is configured to determine the temporal variability of the first scattered light for the first angular range (301a).

C25. The apparatus of any one of C20-C24, wherein the at least one fifth property ([P5]) comprises a magnitude of the second scattered light.

C26. The apparatus of C25 in combination with C15, wherein the computing apparatus (22) is configured to determine the magnitude of the second scattered light for the third angular range (401a).

C27. The apparatus of any one of C20-C25 in combination with any one of C16-C18, wherein the computing apparatus (22) is further configured to determine, based on the third signal (OS2), at least one sixth property ([P6]) representing the transmitted light, and operate the second calculation function (F2) on the at least one fourth property ([P4]), the at least one fifth property ([P5]) and the at least one sixth property ([P6]) to estimate the total particle concentration in the fluid.

C28. The apparatus of C27, wherein the at least one sixth property ([P6]) comprises a temporal variability of the transmitted light.

C29. The apparatus of any preceding clause, wherein the first light beam (300) has a spectral width below 20 nm.

C30. The apparatus of any preceding clause, wherein the first calculation function (F1) comprises a weighted combination of the plurality of first properties ([P1]), as well as any further properties determined by the computing apparatus (40)

C31. The apparatus of C30, wherein the first calculation function (F1) is a linear function.

C32. The apparatus of C30, wherein the first calculation function (F1) is a non-linear function.

C33. A control arrangement for use in the optical detection apparatus of any one of C1-C32.

C34. An apparatus for automated peritoneal dialysis comprising the optical detection apparatus of any one of C1-C32.

C35. A computer-implemented method for determining a concentration of white blood cells in a fluid, said method comprising: obtaining a first signal that represents first scattered light received as a function of time by a light detection arrangement from a first region in the fluid when the first region is illuminated by a first light beam, said first light having a wavelength within a range of 350-575 nm and being directed along a first main direction into the first region; determining, based on the first signal, a plurality of first properties representing the first scattered light; and operating a first calculation function on the plurality of first properties to estimate the concentration of white blood cells in the fluid.

C36. The computer-implemented method of C35, further comprising: obtaining a second signal that represents second scattered light received as a function of time by the light detection arrangement from a second region in the fluid when the second region is illuminated by a second light beam, said second light having a wavelength within a range of 600-1000 nm and being directed along a second main direction into the second region; and determining, based on the second signal, at least one second property representing the second scattered light, wherein the first calculation function is operated on the plurality of first properties and the at least one second property to estimate the concentration of white blood cells in the fluid.

C37. The computer-implemented method of C35 or C36, further comprising: obtaining a third signal representing transmitted light of the first light beam by the first region; and determining, based on the third signal, at least one third property representing the transmitted light, wherein the first calculation function is operated on the plurality of first properties and the at least one third property to estimate the concentration of white blood cells in the fluid.

C38. The computer-implemented method of C36, further comprising estimating a total particle concentration in the fluid, wherein said estimating the total particle concentration comprises: determining, based on the first signal, at least one fourth property representing the first scattered light; determining, based on the second signal, at least one fifth property representing the second scattered light; and operating a second calculation function on the at least one fourth property and the at least one fifth property to estimate the total particle concentration in the fluid.

C39. The computer-implemented method of C38 in combination with C37, wherein said estimating the total particle concentration comprises: determining, based on the third signal, at least one sixth property representing the transmitted light, wherein the second calculation function is operated on the at least one fourth property, the at least one fifth property, and the at least one sixth property to estimate the total particle concentration in the fluid.

C40. A computer-readable medium comprising instructions which when executed by processor circuitry causes the processor circuitry to perform the method of any one of C35-C39.

C41. An optical detection apparatus for determining a concentration of white blood cells in a fluid, said apparatus comprising: a light emitting arrangement (30), which is configured to generate a first light beam with a wavelength within a range of 350-575 nm and arranged to direct the first light beam (300) along a first main direction (310) into a first region (320) in the fluid and to generate a second light beam (400) with a wavelength within a range of 600-1000 nm and is arranged to direct the second light beam (400) along a second main direction (410) into a second region (420) in the fluid; a light detection arrangement (31), which is arranged to detect first scattered light originating from the first light beam (300) in the first region (320) and provide a first signal (OS1; OS11, OS12) that represents the first scattered light as a function of time and to detect second scattered light originating from the second light beam (400) in the second region (420) and provide a second signal (OS1′) that represents the second scattered light as a function of time; and a computing apparatus (22), which is configured to: determine, based on the first signal (OS1; OS11, OS12), at least one fourth property ([P4]) representing the first scattered light; determine, based on the second signal (OS2′), at least one fifth property ([P5]) representing the second scattered light; and operate a second calculation function (F2) on the at least one fourth property ([P4]) and the at least one fifth property ([P5]) to estimate the total particle concentration in the fluid.

C35. A computer-implemented method for determining a total concentration of cells in a fluid, said method comprising: obtaining a first signal that represents first scattered light received as a function of time by a light detection arrangement from a first region in the fluid when the first region is illuminated by a first light beam, said first light having a wavelength within a range of 350-575 nm and being directed along a first main direction into the first region; obtaining a second signal that represents second scattered light received as a function of time by the light detection arrangement from a second region in the fluid when the second region is illuminated by a second light beam, said second light having a wavelength within a range of 600-1000 nm and being directed along a second main direction into the second region; determining, based on the first signal, at least one fourth property representing the first scattered light; determining, based on the second signal, at least one fifth property representing the second scattered light; and operating a second calculation function on the at least one fourth property and the at least one fifth property to estimate the total particle concentration in the fluid.

What is claimed:

1. An optical detection apparatus for determining a concentration of white blood cells in a fluid, said apparatus comprising:
a light emitting arrangement, which is configured to generate a first light beam with a wavelength within a range of 350-575 nanometers (nm) and arranged to direct the first light beam along a first main direction into a first region in the fluid;
a light detection arrangement, which is arranged to detect first scattered light originating from the first light beam in the first region and provide a first signal that represents the first scattered light as a function of time; and
a computing apparatus operably coupled to at least the light detection arrangement and configured to:
determine, based on the first signal, a plurality of first properties representing the first scattered light, and
operate a first calculation function on the plurality of first properties to estimate the concentration of white blood cells in the fluid.

2. The optical detection apparatus of claim 1, wherein the computing apparatus is configured to estimate the concentration of white blood cells in the presence of red blood cells within the fluid.

3. The optical detection apparatus of claim 1, wherein the light detection arrangement is arranged to detect the first scattered light at an angle to the first main direction of the first light beam through the first region, said angle being in a range extending from about 6° to about 35°.

4. The optical detection apparatus of claim 1, wherein the computing apparatus is configured to determine a respective first property to represent an ensemble of signal values within a respective time window of the first signal.

5. The optical detection apparatus of claim 1, wherein the plurality of first properties comprises a magnitude of the first scattered light.

6. The optical detection apparatus of claim 1, wherein the plurality of first properties comprises a temporal variability of the first scattered light.

7. The optical detection apparatus of claim 1, wherein the light detection arrangement is configured to detect the first scattered light in a first angular range and a second angular range, wherein the first angular range differs from the second angular range, and wherein the plurality of first properties comprise a first magnitude of the first scattered light within the first angular range, and a second magnitude of the first scattered light within the second angular range.

8. The optical detection apparatus of claim 7, wherein the first angular range is located within 7°-16° to the first main direction, and the second angular range is located within 16°-35° to the first main direction.

9. The optical detection apparatus of claim 7, wherein computing apparatus is configured to determine a temporal variability of the first scattered light for the second angular range.

10. The optical detection apparatus of claim 1, wherein the light emitting arrangement is configured to generate a second light beam with a wavelength within a range of 600-1000 nm and is arranged to direct the second light beam along a second main direction into a second region in the fluid, wherein the light detection arrangement is arranged to detect second scattered light originating from the second light beam in the second region and provide a second signal that represents the second scattered light as a function of time, and wherein the computing apparatus is configured to determine, based on the second signal, at least one second property representing the second scattered light, and operate the first calculation function on the plurality of first properties and the at least one second property to estimate the concentration of white blood cells.

11. The optical detection apparatus of claim 10, wherein the at least one second property comprises a temporal variability of the second scattered light.

12. The optical detection apparatus of claim 11, wherein the light detection arrangement is configured to detect the second scattered light in a third angular range, which is located within 7°-16° to the second main direction, and wherein the computing apparatus is configured to determine the temporal variability of the second scattered light for the third angular range.

13. The optical detection apparatus of claim 1, wherein the light detection arrangement is further configured to detect transmitted light of the first light beam by the first region and output a third signal representing the transmitted light, and wherein the computing apparatus is configured to determine, based on the third signal, at least one third property representing the transmitted light, and operate the first calculation function on the plurality of first properties and the at least one third property to estimate the concentration of white blood cells in the fluid.

14. The optical detection apparatus of claim 13, wherein the at least one third property comprises a magnitude of the transmitted light.

15. The optical detection apparatus of claim 10, wherein the computing apparatus, to estimate a total particle concentration in the fluid, is further configured to:
determine, based on the first signal, at least one fourth property representing the first scattered light;
determine, based on the second signal, at least one fifth property representing the second scattered light; and operate a second calculation function on the at least one fourth property and the at least one fifth property to estimate the total particle concentration in the fluid, and
wherein the computing apparatus is further configured to estimate a concentration of red blood cells in the fluid as a function of the total particle concentration and the concentration of white blood cells.

16. The optical detection apparatus of claim 15, wherein the at least one fourth property comprises a magnitude of the first scattered light.

17. The optical detection apparatus of claim 15, wherein the at least one fourth property comprises a temporal variability of the first scattered light.

18. The optical detection apparatus of claim 15, wherein the at least one fifth property comprises a magnitude of the second scattered light.

19. The optical detection apparatus of claim 15, wherein the light detection arrangement is further configured to detect transmitted light of the first light beam by the first region and output a third signal representing the transmitted light, and wherein the computing apparatus is configured to determine, based on the third signal, at least one third property representing the transmitted light, and operate the first calculation function on the plurality of first properties and the at least one third property to estimate the concentration of white blood cells in the fluid,
wherein the computing apparatus is further configured to determine, based on the third signal, at least one sixth property representing the transmitted light, and operate the second calculation function on the at least one fourth property, the at least one fifth property and the at least one sixth property to estimate the total particle concentration in the fluid.

20. The optical detection apparatus of claim 19, wherein the at least one sixth property comprises a temporal variability of the transmitted light.
